(19) United States
(12) Patent Application Publication
(10) Pub. No.: US 2005/0272792 A1
(43) Pub. Date: Dec. 8, 2005

(54) Benzimidazoles, process for their preparation and use thereof as medicament

(75) Inventors: Kai Gerlach, Biberach (DE); Roland Pfau, Biberach (DE); Henning Priepke, Warthausen (DE); Annette Schuler-Metz, Ulm (DE); Wolfgang Wienen, Biberach (DE); Sandra Handschuh, Warthausen (DE); Herbert Nar, Ochsenhausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/147,471

(22) Filed: Jun. 7, 2005

(30) Foreign Application Priority Data
Jun. 8, 2004 (DE) 10 2004 027 821

(51) Int. Cl. A61K 31/4184; C07D 403/02; C07D 235/04

(52) U.S. Cl. 514/394; 548/305.4

(57) ABSTRACT

The present invention relates to new substituted benzimidazoles of general formula 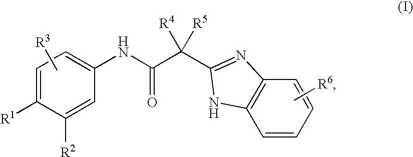
wherein R¹ to R⁶ are defined as in claim 1, the tautomers, the enantiomers, the diastereomers, the mixtures thereof and the salts thereof, particularly the physiologically acceptable salts thereof with inorganic or organic acids or bases, which have valuable properties.

### FIELD OF THE INVENTION

The present invention relates to new substituted benzimidazoles of general formula 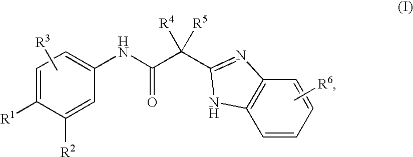
the tautomers, the enantiomers, the diastereomers, the mixtures thereof and the salts thereof, particularly the physiologically acceptable salts thereof with inorganic or organic acids or bases, which have valuable properties.

The compounds of the above general formula I as well as the tautomers, the enantiomers, the diastereomers, the mixtures thereof and the salts thereof, particularly the physiologically acceptable salts thereof with inorganic or organic acids or bases, and their stereoisomers have valuable pharmacological properties, particularly an antithrombotic activity and a factor Xa-inhibiting activity.

The present application thus relates to the new compounds of the above general formula 1, the preparation thereof, the pharmaceutical compositions containing the pharmacologically effective compounds, their preparation and use.

In the above general formula I, in a 1st embodiment
R¹ denotes a 4- to 7-membered cycloalkyleneiminocarbonyl group, wherein
   the cycloalkyleneimino moiety may be substituted in the carbon skeleton by one or two fluorine atoms, one or two C₁₋₃-alkyl, C₂₋₃-alkenyl, C₂₋₃-alkynyl, hydroxy-C₁₋₃-alkyl, C₁₋₃-alkoxy-C₁₋₃-alkyl, phenyl-C₁₋₃-alkyl, heteroaryl-C₁₋₃-alkyl, amino-C₁₋₃-alkyl, C₁₋₅-alkylamino-C₁₋₃-alkyl, di-(C₁₋₅-alkyl)-amino-C₁₋₃-alkyl, a 4- to 7-membered cycloalkyleneimino-C₁₋₃-alkyl, carboxy-C₁₋₃-alkyl, C₁₋₃-alkoxycarbonyl-C₁₋₃-alkyl, aminocarbonyl-C₁₋₃-alkyl, C₁₋₃-alkylaminocarbonyl-C₁₋₃-alkyl, di-(C₁₋₃-alkyl)-aminocarbonyl-C₁₋₃-alkyl, a 4- to 7-membered cycloalkyleneiminocarbonyl-C₁₋₃-alkyl, C₁₋₅-alkoxycarbonylamino-C₁₋₃-alkyl, C₁₋₃-alkyl-carbonylamino-C₁₋₃-alkyl, C₁₋₃-alkylsulphonylamino-C₁₋₃-alkyl,-carboxy, C₁₋₃-alkoxy-carbonyl, aminocarbonyl, C₁₋₃-alkylaminocarbonyl, di-(C₁₋₃-alkyl)-aminocarbonyl, a 4- to 7-membered cycloalkyleneiminocarbonyl, hydroxy, C₁₋₃-alkoxy, amino, C₁₋₃-alkylamino, di-(C₁₋₃-alkyl)-amino, a 4- to 7-membered cycloalkyleneimino, a phenyl or a 5- to 6-membered heteroaryl group, with the proviso that in the substitution of a methylene group adjacent to the imino group two heteroatoms are separated from one another by at least two carbon atoms, and/or
   a methylene group in the 3-position of a 5-membered cycloalkyleneimino group may be replaced by a sulphur atom, a sulphinyl or sulphonyl group, or
   a methylene group in the 4-position of a 6- or 7-membered cycloalkyleneimino group may be replaced by an oxygen or sulphur atom, a carbonyl, sulphinyl or sulphonyl group or by an —NH— group optionally substituted by a C₁₋₃-alkyl, formyl or C₁₋₃-alkylcarbonyl group, while additionally a methylene group adjacent to the nitrogen atom, to which the cycloalkyleneimino group is bound, may be replaced by a carbonyl, sulphinyl or sulphonyl group, with the proviso that
      in the substitution of the previously mentioned 6- to 7-membered cycloalkyleneimino groups, wherein a methylene group is replaced by an oxygen or sulphur atom, a sulphinyl or sulphonyl group, two heteroatoms are separated from one another by at least two carbon atoms,
   a 5- to 7-membered cycloalkenyleneiminocarbonyl group optionally substituted by one or two C₁₋₃-alkyl, amino-C₁₋₃-alkyl, C₁₋₃-alkylamino-C₁₋₃-alkyl, di-(C₁₋₃-alkyl)-amino-C₁₋₃-alkyl, a 4- to 7-membered cyclo-alkyleneimino-C₁₋₃-alkyl, phenyl, phenyl-C₁₋₃-alkyl, heteroaryl, heteroaryl-C₁₋₃-alkyl, aminocarbonyl, C₁₋₃-alkylaminocarbonyl, di-(C₁₋₃-alkyl)-aminocarbonyl or 4- to 7-membered cycloalkyleneiminocarbonyl groups, wherein the double bond is not bound to the imino nitrogen atom,
   a group of general formula 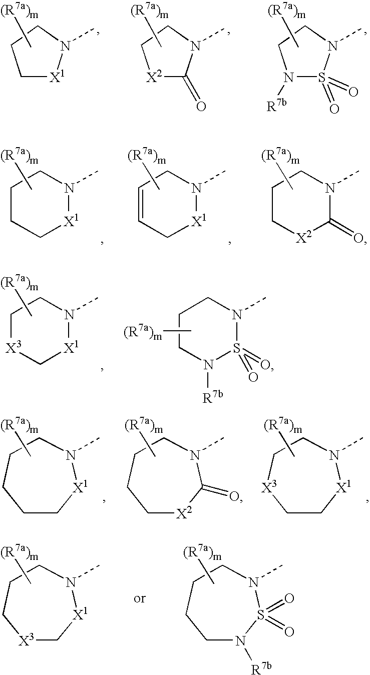
      wherein
   m is the number 1 or 2,
   R^{7a} in each case independently of one another denotes a hydrogen or fluorine atom or a C₁₋₅-alkyl, C₁₋₅-alkoxy-C₁₋₅-alkyl, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl, hydroxy, hydroxy-C₁₋₅-alkyl, C₁₋₅-alkoxy, amino, C₁₋₅-alkylamino, di-(C₁₋₅-alkyl)-amino, or C₁₋₅-alkylcarbonylamino group, while
      in the above-mentioned substituted 5- to 7-membered groups R¹ the heteroatoms F, O or N optionally introduced with R^{7a} as substituents are not separated by precisely one carbon atom from a heteroatom selected from among N, O, S,
   R^{7b} each independently of one another denote a hydrogen atom or a C₁₋₅-alkyl group,
   R^{7c} each independently of one another denote a hydrogen atom, a C₁₋₅-alkyl, C₁₋₅-alkylcarbonyl or C₁₋₅-alkoxycarbonyl group,
   X¹ denotes a carbonyl, thiocarbonyl or sulphonyl group,
   X² denotes an oxygen atom or a —NR^{7b}— group,
   X³ denotes an oxygen or sulphur atom or a —NR^{7c}— group,
      a group of general formula 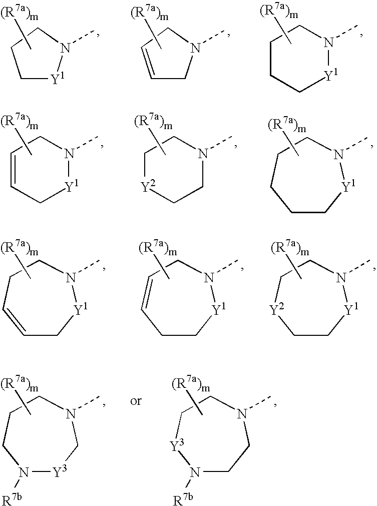
      wherein
         m is the number 1 or 2,
         R⁷a, R⁷b and R⁷c are as hereinbefore defined,
         Y¹ denotes an oxygen atom or a —CH₂—, —CHR^{7b}— or —NR^{7c}— group,
         Y² denotes an oxygen or sulphur atom, an —NR^{7c}— group, and
         Y³ denotes a carbonyl or sulphonyl group,
R² denotes a hydrogen, fluorine, chlorine or bromine atom, a C₁₋₃-alkyl group wherein the hydrogen atoms may be wholly or partly replaced by fluorine atoms, a C₂₋₃-alkenyl, C₂₋₃-alkynyl, C₁₋₃-alkoxy, a mono-, di- or trifluoromethoxy group,
R³ denotes a hydrogen or halogen atom or a C₁₋₃-alkyl group,
R⁴ denotes a hydrogen atom, an amino, C₁₋₅-alkylcarbonylamino, C₁₋₅-alkoxy-carbonylamino, a C₂₋₃-alkenyl or C₂₋₃-alkynyl group,
   a straight-chain or branched C₁₋₅-alkyl group which is optionally substituted by a fluorine atom, a mono-, di- or trifluoromethyl, a nitrile, hydroxy, a C₁₋₅-alkoxy group wherein the hydrogen atoms may be wholly or partly replaced by fluorine atoms, a mercapto, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, a 4- to 7-membered cycloalkyleneimino, C₁₋₅-alkylcarbonylamino, carboxy or C₁₋₅-alkoxycarbonyl group,
   a phenyl or heteroaryl, phenyl-C₁₋₃-alkyl or heteroaryl-C₁₋₃-alkyl group which is optionally mono- or polysubstituted by fluorine, chlorine or bromine atoms, C₁₋₃-alkyl, amino, C₁₋₃-alkylamino, di-(C₁₋₃-alkyl)-amino, hydroxy, C₁₋₄-alkoxy, mono-, di- or trifluoromethoxy, carboxy or C₁₋₃-alkoxycarbonyl group,
R⁵ denotes a hydrogen atom or a C₁₋₃-alkyl group, or
R⁴ and R⁵ together with the carbon atom to which they are bound denote a C₃-₆-cycloalkyl group, wherein
   one of the methylene groups of the C₃₋₆-cycloalkyl group thus formed may be replaced by an oxygen or sulphur atom, an imino, C₁₋₃-alkylimino or acylimino group, and
R⁶ denotes a hydrogen, fluorine, chlorine or bromine atom, a C₁₋₃-alkyl group wherein the hydrogen atoms may be wholly or partly replaced by fluorine atoms, a C₂₋₃-alkenyl or C₂₋₃-alkynyl, a hydroxy, C₁₋₃-alkoxy, trifluoromethoxy, amino, nitro or cyano group,
while, unless otherwise stated, by the term “heteroaryl group” mentioned hereinbefore in the definitions is meant a monocyclic 5- or 6-membered heteroaryl group optionally substituted in the carbon skeleton by a fluorine, chlorine, bromine or iodine atom, a C₁₋₃-alkyl, amino, C₁₋₃-alkylamino, di-(C₁₋₃-alkyl)-amino, C₁₋₃-alkoxy, carboxy, C₁₋₃-alkoxycarbonyl or C₁₋₃-alkoxycarbonylamino group,
wherein
   the 6-membered heteroaryl group contains one, two or three nitrogen atoms and
   the 5-membered heteroaryl group contains an imino group optionally substituted by a C₁₋₃-alkyl or phenyl-C₁₋₃-alkyl group, or an oxygen or sulphur atom, or
   an imino group optionally substituted by a C₁₋₃-alkyl, amino-C₂₋₃-alkyl, C₁₋₃-alkylamino-C₂₋₃-alkyl, di-(C₁₋₃-alkyl)-amino-C₂₋₃-alkyl, a 4- to 7-membered cycloalkyleneimino-C₁₋₃-alkyl or phenyl-C₁₋₃-alkyl group, or an oxygen or sulphur atom and additionally a nitrogen atom or
   an imino group optionally substituted by a C₁₋₃-alkyl or phenyl-C₁₋₃-alkyl group and two or three nitrogen atoms,
   and moreover a phenyl ring optionally substituted by a fluorine, chlorine or bromine atom or a C₁₋₃-alkyl, hydroxy or C₁₋₃-alkoxy group may be fused to the above-mentioned monocyclic heteroaryl groups via two adjacent carbon atoms
   and the bond is effected via a nitrogen atom or a carbon atom of the heterocyclic moiety or a fused-on phenyl ring,
while the alkyl and alkoxy groups contained in the foregoing definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms.

Examples of monocyclic heteroaryl groups are the pyridyl, N-oxy-pyridyl, pyrazolyl, pyridazinyl, pyrimidinyl, pyrazinyl, [1,2,3]triazinyl, [1,3,5]triazinyl, [1,2,4]triazinyl, pyrrolyl, imidazolyl, [1,2,4]triazolyl, [1,2,3]triazolyl, tetrazolyl, furanyl, isoxazolyl, oxazolyl, [1,2,3]oxadiazolyl, [1,2,4]oxadiazolyl, furazanyl, thiophenyl, thiazolyl, isothiazolyl, [1,2,3]thiadiazolyl, [1,2,4]thiadiazolyl or [1,2,5]thiadiazolyl group.

Examples of bicyclic heteroaryl groups are the benzimidazolyl, benzofuranyl, benzo[c]furanyl, benzothiophenyl, benzo[c]thiophenyl, benzothiazolyl, benzo[c]-isothiazolyl, benzo[d]isothiazolyl, benzoxazolyl, benzo[c]isoxazolyl, benzo[d]-isoxazolyl, benzo[1,2,5]oxadiazolyl, benzo[1,2,5]thiadiazolyl, benzo[1,2,3]thia-diazolyl, benzo[d][1,2,3]triazinyl, benzo[1,2,4]triazinyl, benzotriazolyl, cinnolinyl, quinolinyl, isoquinolinyl, quinazolinyl, quinoxalinyl, phthalazinyl, indolyl, isoindolyl or 1-oxa-2,3-diaza-indenyl group.

Examples of the C₁₋₅-alkyl groups mentioned hereinbefore in the definitions are the methyl, ethyl, 1-propyl, 2-propyl, n-butyl, sec-butyl, tert-butyl, 1-pentyl, 2-pentyl or 3-pentyl group.

Examples of the C₁₋₅-alkoxy groups mentioned hereinbefore in the definitions are the methyloxy, ethyloxy, 1-propyloxy, 2-propyloxy, n-butyloxy, sec-butyloxy, tert-butyloxy, 1-pentyloxy, 2-pentyloxy or 3-pentyloxy group.

A 2nd embodiment of the present invention comprises those compounds of general formula 1, wherein
R¹ denotes a 4- to 7-membered cycloalkyleneiminocarbonyl group, wherein
   the cycloalkyleneimino moiety may be substituted in the carbon skeleton by one or two fluorine atoms, one or two C₁₋₃-alkyl, hydroxy-C₁₋₃-alkyl, heteroaryl-C₁₋₃-alkyl, amino-C₁₋₃-alkyl, C₁₋₅-alkylamino-C₁₋₃-alkyl, di-(C₁₋₅-alkyl)-amino-C₁₋₃-alkyl, a 4- to 7-membered cycloalkyleneimino-C₁₋₃-alkyl, carboxy-C₁₋₃-alkyl, C₁₋₃-alkoxycarbonyl-C₁₋₃-alkyl, carboxy, C₁₋₃-alkoxycarbonyl, aminocarbonyl, C₁₋₃-alkylaminocarbonyl, di-(C₁₋₃-alkyl)-aminocarbonyl, a 4- to 7-membered cycloalkyleneiminocarbonyl, hydroxy, C₁₋₃-alkoxy, amino, C₁₋₃-alkylamino, di-(C₁₋₃-alkyl)-amino, a 4- to 7-membered cycloalkyleneimino or a 5- to 6-membered heteroaryl group, with the proviso that in the substitution of a methylene group adjacent to the imino group two heteroatoms are separated from one another by at least two carbon atoms, and/or
   a methylene group in the 3-position of a 5-membered cycloalkyleneimino group may be replaced by a sulphur atom or
   a methylene group in the 4-position of a 6- or 7-membered cycloalkyleneimino group may be replaced by an oxygen or sulphur atom, a carbonyl, sulphinyl or sulphonyl group or by an —NH— group optionally substituted by a C₁₋₃-alkyl, formyl or C₁₋₃-alkylcarbonyl group, with the proviso that
   in the substitution of the previously mentioned 6- to 7-membered cycloalkyleneimino groups, wherein a methylene group is replaced by an oxygen or sulphur atom, a sulphinyl or sulphonyl group, two heteroatoms are separated from one another by at least two carbon atoms,
a 5- to 7-membered cycloalkenyleneiminocarbonyl group optionally substituted by one or two C₁₋₃-alkyl, amino-C₁₋₃-alkyl, C₁₋₃-alkylamino-C₁₋₃-alkyl, di-(C₁₋₃-alkyl)-amino-C₁₋₃-alkyl, a 4- to 7-membered cyclo-alkyleneimino-C₁₋₃-alkyl, heteroaryl, heteroaryl-C₁₋₃-alkyl, aminocarbonyl, C₁₋₃-alkylaminocarbonyl, di-(C₁₋₃-alkyl)-aminocarbonyl or 4- to 7-membered cycloalkyleneiminocarbonyl groups, wherein the double bond is not bound to the imino nitrogen atom,
a group of general formula 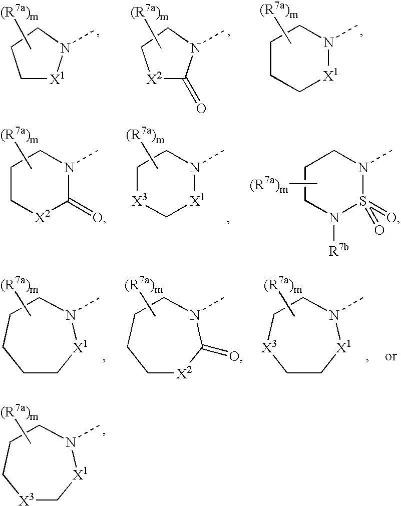
wherein
   m is the number 1 or 2,
   R^{7a} each independently of one another denote a hydrogen or fluorine atom or a C₁₋₅-alkyl, amino-C₁₋₅-alkyl, C₁₋₅-alkylamino-C₁₋₅-alkyl, di-(C₁₋₅-alkyl)-amino-C₁₋₅-alkyl, hydroxy, hydroxy-C₁₋₅-alkyl, C₁₋₅-alkoxy, amino, C₁₋₅-alkylamino or di-(C₁₋₅-alkyl)-amino group, while
      in the above-mentioned substituted 5- to 7-membered groups R¹ the heteroatoms F, O or N optionally introduced with R^{7a} as substituents are not separated by precisely one carbon atom from a heteroatom selected from among N, O, S,
   R^{7b} each independently of one another denote a hydrogen atom or a C₁₋₅-alkyl group,
   R^{7c} each independently of one another denote a hydrogen atom, a C₁₋₅-alkyl or C₁₋₅-alkylcarbonyl group,
   X¹ denotes a carbonyl or sulphonyl group,
   X² denotes an oxygen atom or a —NR^{7b}— group,
   X³ denotes an oxygen or sulphur atom or a —NR^{7c}— group,
      a group of general formula 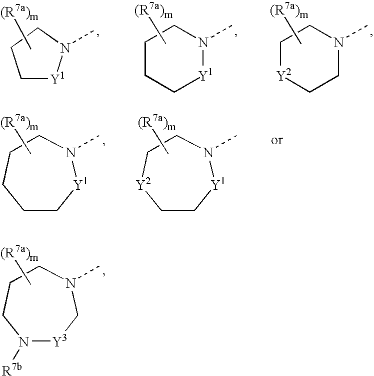
   wherein
      m is the number 1 or 2,
      R^{7a}, R^{7b} and R^{7c} are as hereinbefore defined,
      Y¹ denotes an oxygen atom or a —CH₂—, —CHR^{7b}— or —NR^{7c}— group,
      Y² denotes an oxygen or sulphur atom, an —NR^{7c} group, and
      Y³ denotes a carbonyl or sulphonyl group,
R² denotes a hydrogen, fluorine, chlorine or bromine atom, a C₁₋₃-alkyl group wherein the hydrogen atoms may be wholly or partly replaced by fluorine atoms, a C₂₋₃-alkenyl, C₂₋₃-alkynyl, C₁₋₃-alkoxy, a mono-, di- or trifluoromethoxy group,
R³ denotes a hydrogen or fluorine, chlorine or bromine atom or a C₁₋₃-alkyl group,
R⁴ denotes a hydrogen atom, an amino, C₁₋₅-alkylcarbonylamino, C₁₋₅-alkoxycarbonylamino, a C₂₋₃-alkenyl or C₂₋₃-alkynyl group,
   a straight-chain or branched C₁₋₅-alkyl group which is optionally substituted by a fluorine atom, a mono-, di- or trifluoromethyl, a hydroxy, a C₁₋₅-alkoxy group wherein the hydrogen atoms may be wholly or partly replaced by fluorine atoms, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, a 4- to 7-membered cycloalkyleneimino, carboxy or C₁₋₅-alkoxycarbonyl group,
   a phenyl, heteroaryl or heteroaryl-C₁₋₃-alkyl group which is optionally mono- or polysubstituted by fluorine, chlorine or bromine atoms, C₁₋₃-alkyl, amino, C₁₋₃-alkylamino, di-(C₁₋₃-alkyl)-amino, hydroxy, C₁₋₄-alkoxy, mono-, di- or trifluoromethoxy, carboxy or C₁₋₃-alkoxycarbonyl group,
R⁵ denotes a hydrogen atom or a C₁₋₃-alkyl group, or
R⁴ and R⁵ together with the carbon atom to which they are bound denote a C₃₋₆-cycloalkyl group, wherein
   one of the methylene groups of a C₄₋₆-cycloalkyl group thus formed may be replaced by an oxygen or sulphur atom, an imino, C₁₋₃-alkylimino or acylimino group, and
R⁶ denotes a fluorine, chlorine or bromine atom, a methyl group wherein the hydrogen atoms may be wholly or partly replaced by fluorine atoms, an ethenyl or ethynyl, a methoxy or cyano group,
while, unless otherwise stated, by the term “heteroaryl group” mentioned hereinbefore in the definitions is meant a pyridyl, pyrazolyl, pyridazinyl, pyrimidinyl, pyrazinyl, [1,3,5]triazinyl, pyrrolyl, imidazolyl, tetrazolyl, furanyl, isoxazolyl, oxazolyl, thiophenyl, thiazolyl, isothiazolyl group optionally substituted in the carbon skeleton by a fluorine, chlorine, bromine or iodine atom, a C₁₋₃-alkyl, amino, C₁₋₃-alkylamino, di-(C₁₋₃-alkyl)-amino, C₁₋₃-alkoxy, carboxy, C₁₋₃-alkoxycarbonyl or C₁₋₃-alkoxycarbonylamino group,
while the alkyl and alkoxy groups contained in the foregoing definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
the tautomers, the diastereomers, the enantiomers, the mixtures thereof and the salts thereof.
   A 3rd embodiment of the present invention comprises those compounds of general formula I, wherein
   R¹ denotes a 5- or 6-membered cycloalkyleneiminocarbonyl group, wherein
      the cycloalkyleneimino moiety may be substituted in the carbon skeleton by one or two C₁₋₃-alkyl groups, and/or
      a methylene group in the 3-position of a 5-membered cycloalkyleneimino group may be replaced by a sulphur atom, or
      a methylene group in the 4-position of a 6- or 7-membered cycloalkyleneimino group may be replaced by an oxygen or sulphur atom or by an —NH— group optionally substituted by a C₁₋₃-alkyl, formyl or C₁₋₃-alkylcarbonyl group,
      a 5- or 6-membered cycloalkenyleneiminocarbonyl group optionally substituted by one or two C₁₋₃-alkyl groups, wherein the double bond is not bound to the imino nitrogen atom,
   a group of general formula 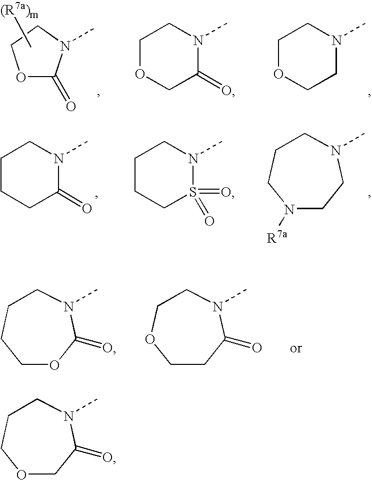
      wherein
      m is the number 1 or 2 and
      R^{7a} each independently of one another denote a hydrogen or a C₁₋₅-alkyl group,
R² denotes a hydrogen, chlorine or bromine atom, a methyl group wherein the hydrogen atoms may be wholly or partly replaced by fluorine atoms, a methoxy, a mono-, di- or trifluoromethoxy group,
   R³ denotes a hydrogen atom,
   R⁴ denotes a hydrogen atom, an amino, C₁₋₅-alkylcarbonylamino, C₁₋₅-alkoxy-carbonylamino, a C₂₋₃-alkenyl or C₂₋₃-alkynyl group,
      a straight-chain or branched C₁₋₄-alkyl group which is optionally substituted by a mono-, di- or trifluoromethyl, a hydroxy, a methoxy group wherein the hydrogen atoms may be wholly or partly replaced by fluorine atoms, C₁₋₂-alkylsulphanyl, C₁₋₂-alkylsulphinyl, C₁₋₂-alkylsulphonyl, amino, C₁₋₂-alkylamino, di-(C₁₋₂-alkyl)-amino, a 4- to 6-membered cycloalkyleneimino, carboxy or methoxycarbonyl group,
      a phenyl, pyridyl or thiophenyl group,
   R⁵ denotes a hydrogen atom or a C₁₋₂-alkyl group, or
R⁴ and R⁵ together with the carbon atom to which they are bound denote a C₃₋₆-cycloalkyl group, preferably a C₄₋₆-cycloalkyl group, wherein
   one of the methylene groups of a C₃₋₆-cycloalkyl group or C₄₋₆-cycloalkyl group thus formed may be replaced by an oxygen or sulphur atom, an imino, C₁₋₃-alkylimino or acylimino group, and
R⁶ denotes a chlorine or bromine atom or an ethynyl group,
while the alkyl and alkoxy groups contained in the foregoing definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
the tautomers, the diastereomers, the enantiomers, the mixtures thereof and the salts thereof.

The following preferred compounds of general formula I will now be mentioned by way of example:
(1) 2-(5-chloro-1H-benzimidazol-2-yl)-N-[3-methyl4-(morpholin-3-on-4-yl)-phenyl]-2-phenyl-acetamide,
(2) 1-(5-chloro-1H-benzimidazol-2-yl)-N-[3-methyl-4-(morpholin-3-on-4-yl)-phenyl]-cyclopropanecarboxamide,
(3) 2-(5-chloro-1H-benzimidazol-2-yl)-N-[3-methyl-4-(morpholin-3-on-4-yl)-phenyl]-propionamide,
(4) 2-(5-chloro-1H-benzimidazol-2-yl)-N-[3-methyl-4-(pyrrolidin-1-ylcarbonyl)-phenyl]-propionamide,
(5) 2-(5-chloro-1H-benzimidazol-2-yl)-N-[3-methyl-4-(pyrrolidin-1-ylcarbonyl)-phenyl]-2-amino-acetamide,
(6) 2-(5-chloro-1H-benzimidazol-2-yl)-N-[3-methyl-4-(pyrrolidin-1-ylcarbonyl)-phenyl]-2-tert.butoxycarbonylamino-acetamide,
(7) 2-(5-chloro-1H-benzimidazol-2-yl)-N-[3-methyl-4-(pyrrolidin-1-ylcarbonyl)-phenyl]-2-phenyl-acetamide,
(8) 2-(5-chloro-1H-benzimidazol-2-yl)-N-[3-methyl-4-(pyrrolidin-1-ylcarbonyl)-phenyl]-pent-4-enoic acid amide,
(9) 4-(5-chloro-1H-benzimidazol-2-yl)-N-[3-methyl4-(morpholin-3-on-4-yl)-phenyl]-1-methyl-piperidine-4-carboxamide,
(10) 2-(5-chloro-1H-benzimidazol-2-yl)-N-[3-methyl4-(2,5-dihydro-pyrrol-1-yl-carbonyl)-phenyl]-2-(pyrid-3-yl)-acetamide,
(11) 2-(5-chloro-1H-benzimidazol-2-yl)-N-[3-methyl-4-(thiazolidin-3-ylcarbonyl)-phenyl]-propionamide,
(12) 2-(5-chloro-1H-benzimidazol-2-yl)-N-[3-methyl-4-(morpholin-4-ylcarbonyl)-phenyl]-propionamide,
(13) 2-(5-chloro-1H-benzimidazol-2-yl)-N-[3-methyl-4-(pyrrolidin-1-ylcarbonyl)-phenyl]4-phenyl-butanoic acid amide,
(14) 2-(5-chloro-1H-benzimidazol-2-yl)-N-[3-methyl-4-(pyrrolidin-1-ylcarbonyl)-phenyl]-5-dimethylamino-pentanoic acid amide,
(15) 2-(5-chloro-1H-benzimidazol-2-yl)-N-[3-methyl-4-(pyrrolidin-1-ylcarbonyl)-phenyl]-4-methyl-pentanoic acid amide,
(16) 2-(5-chloro-1H-benzimidazol-2-yl)-N-[3-methyl-4-(pyrrolidin-1-ylcarbonyl)-phenyl]-2-methyl-propanoic acid amide,
(17) 4-(5-chloro-1H-benzimidazol-2-yl)-N-[3-methyl-4-(morpholin-3-on-4-yl)-phenyl]-tetrahydropyran-4-carboxamide,
(18) 2-(5-chloro-1H-benzimidazol-2-yl)-N-[3-methyl-4-(pyrrolidin-1-ylcarbonyl)-phenyl]-4-hydroxy-butanoic acid amide,
(19) 2-(5-chloro-1H-benzimidazol-2-yl)-N-[3-methoxy-4-(pyrrolidin-1-ylcarbonyl)-phenyl]-4-methoxy-butanoic acid amide,
(20) 1-(5-chloro-1H-benzimidazol-2-yl)-N-[3-methyl-4-(morpholin-3-on-4-yl)-phenyl]-cyclopentanecarboxamide,
(21) 2-(5-chloro-1H-benzimidazol-2-yl)-N-[3-chloro-4-(morpholin-3-on-4-yl)-phenyl]-2-methyl-propanecarboxamide,
(22) 2-(5-bromo-1H-benzimidazol-2-yl)-N-[3-trifluoromethyl-4-(piperidin-2-on-1-yl)-phenyl]-propionamide,
(23) 2-(5-bromo-1H-benzimidazol-2-yl)-N-[3-chloro4-(4,4-dimethyl-oxazolidin-2-on-3-yl)-phenyl]-propionamide,
(24) 2-(5-chloro-1H-benzimidazol-2-yl)-N-[3-chloro-4([1,3]oxazepan-2-on-3-yl)-phenyl]-2-propionamide,
(25) 2-(5-chloro-1H-benzimidazol-2-yl)-N-[3-chloro-4([1,4]oxazepan-5-on-4-yl)-phenyl]-2-propionamide,
(26) 2-(5-chloro-1H-benzimidazol-2-yl)-N-[3-chloro-4-(morpholin-3-on-4-yl)-phenyl]-4-methoxy-butanecarboxamide,
(27) 2-(5-chloro-1H-benzimidazol-2-yl)-N-[3-methyl-4-(1,1-dioxo-[1,2]thiazinan-2-yl)-phenyl]-propionamide,
(28) 2-(5-chloro-1H-benzimidazol-2-yl)-N-[3-chloro-4-(morpholin-3-on-4-yl)-phenyl]-4-hydroxy-butanecarboxamide,
(29) 2-(5-chloro-1H-benzimidazol-2-yl)-N-[3-methyl-4-(2-methyl-pyrrolidin-1-yl-carbonyl)-phenyl]-propionamide,
(30) 2-(5-chloro-1H-benzimidazol-2-yl)-N-[3-trifluoromethyl-4-(pyrrolidin-1-ylcarbonyl)-phenyl]-2-thiophen-3-yl-acetamide,
(31) 2-(5-chloro-1H-benzimidazol-2-yl)-N-[3-chloro-4-(morpholin-3-on-4-yl)-phenyl]-4-methylsulphanyl-butanecarboxamide,
(32) 2-(5-chloro-1H-benzimidazol-2-yl)-N-[3-chloro-4-(morpholin-3-on-4-yl)-phenyl]-4-methylsulphonyl-butanecarboxamide,
(33) 2-(5-chloro-1H-benzimidazol-2-yl)-N-[3-methyl-4-(pyrrolidin-1-ylcarbonyl)-phenyl]-4-carboxy-butanoic acid amide,
(34) 2-(5-chloro-1H-benzimidazol-2-yl)-N-[3-methyl-4-(pyrrolidin-1-ylcarbonyl)-phenyl]-4-methoxycarbonyl-butanoic acid amide,
(35) 2-(5-chloro-1H-benzimidazol-2-yl)-N-[3-chloro-4-(4-methyl-[1,4]-diazepan-1-yl)-phenyl]-propanoic acid amide,
(36) 2-(5-chloro-1H-benzimidazol-2-yl)-N-[3-methyl-4-(4-methyl-[1,4]-diazepan-1-yl)-phenyl]-2-methylpropanoic acid amide,
(37) 2-(5-chloro-1H-benzimidazol-2-yl)-N-[3-methyl-4-(morpholin-1-yl)-phenyl]-2-methyl-pentanoic acid amide,
(38) 2-(5-bromo-1H-benzimidazol-2-yl)-N-[3-methyl-4-(morpholin-3-on-4-yl)-phenyl]-2-methyl-propanecarboxamide,
(39) 2-(5-ethynyl-1H-benzimidazol-2-yl)-N-[3-methyl-4-(morpholin-3-on-4-yl)-phenyl]-2-methyl-propanecarboxamide, and
(40) 2-(5-bromo-1H-benzimidazol-2-yl)-N-[3-methyl-4-(morpholin-3-on-4-yl)-phenyl]aminocarbonyl-tetrahydrofuran,
   the tautomers, the diastereomers, the enantiomers, the mixtures thereof and the salts thereof.

According to the invention the compounds of general formula I are obtained by methods known per se, for example by the following methods:
(a) In order to prepare a compound of general formula 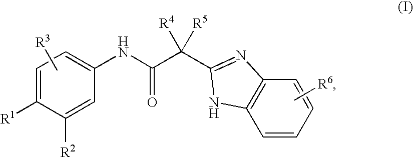
   wherein R¹ to R⁶ are as hereinbefore defined:
acylation of a carboxylic acid or a reactive carboxylic acid derivative of general formula 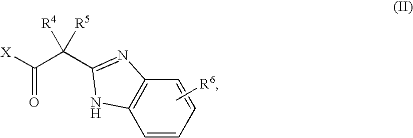
   wherein R⁴ to R⁶ are as hereinbefore defined and X denotes a hydroxy, C₁₋₄-alkoxy group or a halogen atom or the group C(O)X denotes an activated form of a carboxylic acid, such as for example a carboxylic acid anhydride, with a compound of general formula 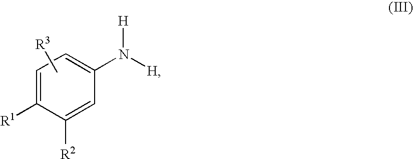
   wherein R¹ to R³ are as hereinbefore defined.

The acylation is conveniently carried out with the free acid or an ester, optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, triethylamine, N,N′-dicyclohexylcarbodiimide, N,N′-dicyclohexylcarbodiimide/N-hydroxysuccinimide or 1-hydroxy-benzotriazole, O-(benzotriazol-1-yl)-N, N, N′, N′-tetramethyluronium tetrafluoroborate, propanephosphonic acid cycloanhydride, N,N′-carbonyldiimidazole or N,N′-thionyldiimidazole or triphenylphosphine/carbon tetrachloride, in a solvent or mixture of solvents such as methylene chloride, chloroform, carbon tetrachloride, ether, tetrahydrofuran, dioxane, benzene, toluene, acetonitrile, dimethylformamide or sulpholane and optionally with the addition of an auxiliary base such as for example N-methylmorpholine, triethylamine, diisopropylethylamine, potassium carbonate or sodium hydrogen carbonate, at temperatures between −20 and 200° C., but preferably at temperatures between −10 and 100° C., or also thermally, optionally with additional microwave irradiation in a solvent or mixture of solvents such as dimethylformamide, sulpholane, dimethylsulphoxide, N-methylpyrrolidinone, toluene or xylylene at temperatures between 100 and 250° C., but preferably between 130 and 200° C.

The acylation may, however, also be carried out with a corresponding halide or anhydride in a solvent such as methylene chloride, chloroform, carbon tetrachloride, ether, tetrahydrofuran, dioxane, benzene, toluene, acetonitrile, dimethylformamide or sulpholane, optionally in the presence of an inorganic or organic base at temperatures between −20 and 200° C., but preferably at temperatures between −10 and 160° C.

Other methods of amide coupling are described, for example, in P. D. Bailey, I. D. Collier, K. M. Morgan in “Comprehensive Functional Group Interconversions”, Vol. 5, page 257ff., Pergamon 1995.
b) In order to prepare a compound of general formula 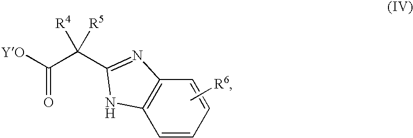
   wherein R⁴, R⁵ and R⁶ are as hereinbefore defined and Y′ denotes a hydrogen atom or a carboxyl-protective group:
cyclisation of a compound of general formula 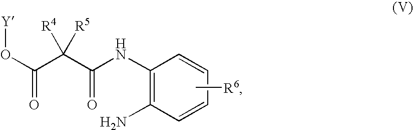
   optionally formed in the reaction mixture, wherein R⁴ to R⁶ are as hereinbefore defined and Y′ denotes the hydrogen atom or a carboxyl protecting group as defined hereinafter, and any protecting group used is then cleaved.

The cyclisation is conveniently carried out in a solvent or mixture of solvents such as ethanol, isopropanol, glacial acetic acid, benzene, chlorobenzene, toluene, xylene, glycol, glycol monomethyl ether, diethyleneglycol dimethyl ether, sulpholane, dimethylformamide or tetralin, dimethylsulphoxide, methylene chloride, chloroform, tetrachloromethane, for example at temperatures between 0 and 250° C., but preferably between 20 and 100° C., optionally in the presence of a condensing agent such as phosphorus oxychloride, propanephosphonic acid cycloanhydride, thionyl chloride, sulphuryl chloride, sulphuric acid, p-toluenesulphonic acid, methanesulphonic acid, hydrochloric acid, phosphoric acid, polyphosphoric acid, acetic acid, acetic anhydride, N,N-dicyclohexyl carbodiimide and/or optionally also in the presence of a base such as for example diisopropylethylamine, triethylamine, potassium carbonate, potassium ethoxide or potassium tert. butoxide. The cyclisation may, however, also be carried out without a solvent and/or condensing agent.
c) The compounds of general formula I may, however, also be synthesised as illustrated in the following Diagram: 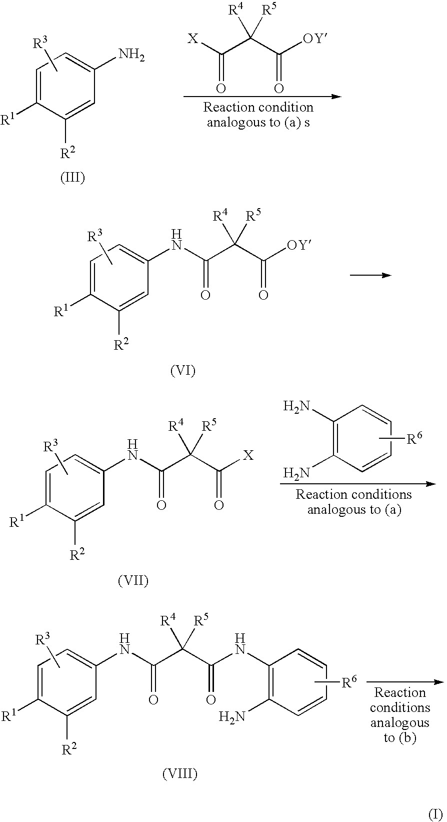
   wherein R¹ to R⁶ are as hereinbefore defined, Y′ denotes the hydrogen atom or a carboxyl protecting group such as a trimethylsilyl, methyl, ethyl, propyl, isopropyl, butyl, isobutyl, tert.-butyl or benzyl group and X denotes a hydroxy or C₁₋₄-alkoxy group.

The first and third reaction steps are carried out by amide formation with an activated carboxylic acid derivative as mentioned under (a).

If desired the conversion of a protected carboxylic acid of general formula VI into a free carboxylic acid of general formula VII is carried out hydrolytically, for example, in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C., or a benzyl, methoxybenzyl or benzyloxycarbonyl group may also be cleaved hydrogenolytically, for example, with hydrogen in the presence of a catalyst such as palladium/charcoal in a solvent or mixture of solvents such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and under a hydrogen pressure of 1 to 7 bar, but preferably 1 to 5 bar.

The cyclisation of a compound of general formula VIII to form a compound of general formula I is carried out as described in step (b).

The compounds of general formulae III and V used as starting materials, some of which are known from the literature, are obtained by methods known from the literature. Moreover, their preparation is described in the Examples.

The preparation of compounds of general formulae V and VIII and their cyclisation to form the derivatives II and I may be carried out, for example, analogously to K. Maekawa, J. Ohtani, Agr. Biol. Chem. 1976, 40, 791-799.

Thus, for example, a compound of general formula V is obtained by acylation of a corresponding o-diamino compound with a corresponding reactive malonic acid derivative.

Compounds of general formula III may also be prepared, for example, analogously to the methods described in the patents WO 02/062748, WO 03/000256 or WO 2004/035039.

Compounds of general formula VI may also be prepared, for example, analogously to the methods described in M. Kawai et al. J. Med. Chem. 1982, 25, 397.

In the reactions described above any reactive groups present such as hydroxy, carboxy, amino, alkylamino or imino groups may be protected during the reaction by conventional protective groups which are cleaved again after the reaction.

For example a suitable protective group for a hydroxy group is the methoxy, benzyloxy, trimethylsilyl, acetyl, benzoyl, tert.-butyl, trityl, benzyl or tetrahydro-pyranyl group,

a suitable protective group for a carboxyl group is the trimethylsilyl, methyl, ethyl, propyl, isopropyl, butyl, isobutyl, tert.-butyl or benzyl group and

a suitable protective group for an amino, alkylamino or imino group is the acetyl, trifluoroacetyl, benzoyl, ethoxycarbonyl, tert.-butoxycarbonyl, benzyloxycarbonyl, benzyl, methoxybenzyl or 2,4-dimethoxybenzyl group and additionally a suitable protective group for the amino group is the phthalyl group.

Other protective groups and their cleaving are described in T. W. Greene, P. G. M. Wuts, “Protective Groups in Organic Synthesis”, Wiley, 1991 and 1999.

Any protective group used is optionally subsequently cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 10 and 50° C.

A benzyl, methoxybenzyl or benzyloxycarbonyl group, however, is cleaved by hydrogenolysis, for example, e.g. with hydrogen in the presence of a catalyst such as palladium/charcoal in a solvent such as methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and under a hydrogen pressure of 1 to 7 bar, but preferably 1 to 5 bar.

A methoxybenzyl group may also be cleaved in the presence of an oxidising agent such as cerium(IV)ammonium nitrate in a solvent such as methylene chloride, acetonitrile or acetonitrile/water at temperatures between 0 and 50° C., but preferably at ambient temperature.

A methoxy group is conveniently cleaved in the presence of boron tribromide in a solvent such as methylene chloride at temperatures between −35 and −25° C.

A 2,4-dimethoxybenzyl group, however, is preferably cleaved in trifluoroacetic acid in the presence of anisole.

A tert.-butyl or tert.-butyloxycarbonyl group is preferably cleaved by treatment with an acid such as trifluoroacetic acid or hydrochloric acid, optionally using a solvent such as methylene chloride, dioxane or ether.

A phthalyl group is preferably cleaved in the presence of hydrazine or a primary amine such as methylamine, ethylamine or n-butylamine in a solvent such as methanol, ethanol, isopropanol, toluene/water or dioxane at temperatures between 20 and 50° C.

An allyloxycarbonyl group is cleaved by treatment with a catalytic amount of tetrakis-(triphenylphosphine)-palladium(0), preferably in a solvent such as tetrahydrofuran and preferably in the presence of an excess of a base such as morpholine or 1,3-dimedone at temperatures between 0 and 100° C., preferably at ambient temperature and under inert gas, or by treatment with a catalytic amount of tris-(triphenylphosphine)-rhodium(I)chloride in a solvent such as aqueous ethanol and optionally in the presence of a base such as 1,4-diazabicyclo[2.2.2]octane at temperatures between 20 and 70° C.

Moreover, the compounds of general formula I obtained may be resolved into their enantiomers and/or diastereomers.

Thus, for example, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical enantiomers and compounds of general formula I with at least 2 asymmetric carbon atoms may be resolved into their diastereomers on the basis of their physical-chemical differences using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be, for example, (+) or (−)-menthol and an optically active acyl group in amides, for example, may be a (+)- or (−)-menthyloxycarbonyl.

Furthermore, the compounds of formula I obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

Moreover, if the new compounds of formula I contain a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, cyclohexylamine, ethanolamine, diethanolamine and triethanolamine.

As already mentioned hereinbefore, the compounds of general formula I and the tautomers, enantiomers, diastereomers and physiologically acceptable salts thereof have valuable pharmacological properties, particularly an antithrombotic activity which is preferably based on an effect on thrombin or factor Xa, for example on a thrombin-inhibiting or factor Xa-inhibiting activity, on a prolonging effect on the aPTT time and on an inhibitory effect on related serine proteases such as e.g. urokinase, factor Vlla, factor IX, factor XI and factor XII.

The compounds listed in the Experimental Section were investigated for their effect on the inhibition of factor Xa as follows:

Method:

Enzyme-kinetic measurement with chromogenic substrate. The quantity of p-nitroaniline (pNA) released from the colourless chromogenic substrate by human factor Xa is determined photometrically at 405 nm. It is proportional to the activity of the enzyme used. The inhibition of the enzyme activity by the test substance (in relation to the solvent control) is determined at various concentrations of test substance and from this the IC₅₀ is calculated, as the concentration which inhibits the factor Xa used by 50%.

Material:

Tris(hydroxymethyl)-aminomethane buffer (100 mMol) and sodium chloride (150 mMol), pH 8.0 plus 1 mg/ml Human Albumin Fraction V, protease-free

Factor Xa (Calbiochem), spec. activity: 217 IU/mg, final concentration: 7 IU/mI for each reaction mixture

Substrate S 2765 (Chromogenix), final concentration: 0.3 mM/I (1 KM) for each reaction mixture

Test substance: final concentration 100, 30, 10, 3, 1, 0.3, 0.1, 0.03, 0.01, 0.003, 0.001 μMol/l

Procedure:

10 μl of a 23.5-times concentrated starting solution of the test substance or solvent (control), 175 μl of TRIS/HSA buffer and 25 μl of a 65.8 U/L Factor Xa working solution are incubated for 10 minutes at 37° C. After the addition of 25 μl of S 2765 working solution (2.82 mMol/l) the sample is measured in a photometer (SpectraMax 250) at 405 nm for 600 seconds at 37° C.

Evaluation:

1. Determining the maximum increase (deltaOD/minutes) over 21 measuring points.

2. Determining the % inhibition based on the solvent control.

3. Plotting a dosage/activity curve (% inhibition vs substance concentration).

4. Determining the IC₅₀ by interpolating the X-value (substance concentration) of the dosage/activity curve at Y=50% inhibition.

All the compounds tested had an IC₅₀ value of less than 10 μmol/L.

The compounds prepared according to the invention are generally well tolerated.

In view of their pharmacological properties the new compounds and the physiologically acceptable salts thereof are suitable for the prevention and treatment of venous and arterial thrombotic diseases, such as for example the prevention and treatment of deep leg vein thrombosis, for preventing reocclusions after bypass operations or angioplasty (PT(C)A), and occlusion in peripheral arterial diseases, and for preventing and treating pulmonary embolism, disseminated intravascular coagulation, for preventing and treating coronary thrombosis, for preventing stroke and the occlusion of shunts. In addition, the compounds according to the invention are suitable for antithrombotic support in thrombolytic treatment, such as for example with alteplase, reteplase, tenecteplase, staphylokinase or streptokinase, for preventing long-term restenosis after PT(C)A, for the prevention and treatment of ischaemic incidents in patients with all forms of coronary heart disease, for preventing metastasis and the growth of tumours and inflammatory processes, e.g. in the treatment of pulmonary fibrosis, for preventing and treating rheumatoid arthritis, for preventing and treating fibrin-dependent tissue adhesions and/or the formation of scar tissue and for promoting wound healing processes. The new compounds and the physiologically acceptable salts thereof may be used therapeutically in conjunction with acetylsalicylic acid, with inhibitors of platelet aggregation such as fibrinogen receptor antagonists (e.g. abciximab, eptifibatide, tirofiban, roxifiban), with physiological activators and inhibitors of the clotting system and the recombinant analogues thereof (e.g. Protein C, TFPI, antithrombin), with inhibitors of ADP-induced aggregation (e.g. clopidogrel, ticlopidine), with P₂T receptor antagonists (e.g. cangrelor) or with combined thromboxane receptor antagonists/synthetase inhibitors (e.g. terbogrel).

The dosage required to achieve such an effect is appropriately 0.01 to 3 mg/kg, preferably 0.03 to 1.0 mg/kg by intravenous route, and 0.03 to 30 mg/kg, preferably 0.1 to 10 mg/kg by oral route, in each case administered 1 to 4 times a day.

For this purpose, the compounds of formula I prepared according to the invention may be formulated, optionally together with other active substances, with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof, to produce conventional galenic preparations such as plain or coated tablets, capsules, powders, suspensions or suppositories.

The Examples that follow are intended to illustrate the invention, without restricting its scope:

Experimental Section

As a rule, melting points, IR, UV, ¹H-NMR and/or mass spectra have been obtained for the compounds prepared. Unless otherwise stated, R_{f} values were determined using ready-made silica gel 60 F₂₅₄ TLC plates (E. Merck, Darmstadt, Item no. 1.05714) without chamber saturation. The R_{f} values given under the heading Alox were determined using ready-made aluminium oxide 60 F₂₅₄ TLC plates (E. Merck, Darmstadt, Item no.1.05713) without chamber saturation. The R_{f} values given under the heading Reversed-phase-8 were determined using ready-made RP-8 F_{254S}, TLC plates (E. Merck, Darmstadt, Item no. 1.15684) without chamber saturation. The ratios given for the eluants refer to units by volume of the solvents in question. For chromatographic purification silica gel made by Millipore (MATREX™, 35-70 my) was used. Unless more detailed information is provided as to the configuration, it is not clear whether the products are pure stereoisomers or mixtures of enantiomers and diastereomers.

The following abbreviations are used in the descriptions of the experiments:
Boc tert.-butoxycarbonyl
DIPEA N-ethyl-diisopropylamine
DMSO dimethylsulphoxide
DMF N,N-dimethylformamide
sat. saturated
i. vac. in vacuo
conc. concentrated
NMM N-methyl-morpholine
NMP N-methyl-pyrrolidin-2-one
PPA propanephosphonic acid cycloanhydride
quant. quantitative
R_{f} retention factor
Rₜ retention time
rac. racemic
TBTU O-(benzotriazol-1-yl)-N,N,N′,N′-tetramethyluronium tetrafluoroborate
TEA triethylamine
TFA trifluoroacetic acid
THF tetrahydrofuran
tert. tertiary

### EXAMPLE 1

### 2-(5-chloro-1H-benzimidazol-2-yl)-N-[3-methyl-4-(morpholin-3-on-4-yl)-phenyl]-2-phenyl-acetamide

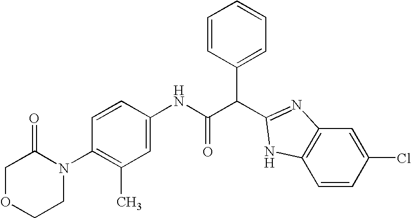 (a) ethyl 2-(5-chloro-1H-benzimidazol-2-yl)-2-phenyl-acetate
i) 4.00 g (190 mmol) ethyl-2-phenylmalonate and 2.88 g (20 mmol) 4-chloro-1,2-phenylenediamine are dissolved in 50 ml THF and at 0° C. combined with 14 ml 50% PPA solution in ethyl acetate (24 mmol) and 6.0 ml TEA (43 mmol). After 30 min the mixture is heated to ambient temperature and stirred for 4 h. Then it is evaporated down i. vac. and the crude product is purified by chromatography (silica gel; eluant: methylene chloride->methylene chloride/ethanol 92:8).
ii) The reaction product is stirred for 4 h in glacial acetic acid at 60° C., then it is evaporated down i. vac. and purified by chromatography (silica gel; eluant: methylene chloride->methylene chloride/ethanol 95:5).

Yield: 3.17 g (53%) R_{f} value: 0.65 (silica gel; dichloromethane/ethanol=9:1) C₁₇H₁₅ClN₂O₂ (314.77) Mass spectrum: (M−H)⁻=313/315 (chlorine isotope) (M+H)⁺=315/317 (chlorine isotope)

(b) 2-(5-chloro-1H-benzimidazol-2-yl)-N-[3-methyl4-(morpholin-3-on-4-yl)-phenyl]-2-phenyl-acetamide

0.470 g (1.49 mmol) ethyl 2-(5-chloro-1H-benzimidazol-2-yl)-2-phenyl-acetate and 0.308 g (1.49 mmol) 3-methyl-4-(morpholin-3-on-4-yl)-aniline in 1.0 ml NMP are heated in the microwave for 10 min. each at 100° C., 150° C. and 180° C. Then the reaction mixture is poured into water, extracted twice with ethyl acetate, the organic phase is washed with sat. NaCl solution, dried with Na₂SO₄ and evaporated down i. vac. The crude product is purified by chromatography (silica gel; eluant: methylene chloride->methylene chloride/ethanol 97:3) and triturated with water. The white precipitate was suction filtered and dried at 40° C.

Yield: 0.390 g (55%) R_{f}value: 0.48 (silica gel; dichloromethane/ethanol=9:1) C₂₆H₂₃ClN₄O₃ (474.94) Mass spectrum: (M−H)⁻=473/475 (chlorine isotope) (M+H)⁺=475/477 (chlorine isotope)

The following was prepared analogously to the sequence described in Example 1:

| | Structural formula | | | |
|---|---|---|---|---|
| No. | Name | Yield* | Mass peak(s) | R_{f} value |
| 07 | 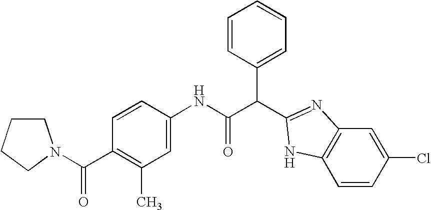 | 50% | (M + H)⁺ = 473/475 (chlorine isotope) | 0.38 (silica gel, CH₂Cl₂/ —C₂H₅OH 9:1) |

2-(5-chloro-1H-benzimidazol-2-yl)-N-[3-methyl-4-(pyrrolidin-1-ylcarbonyl)-phenyl]-2-phenyl-acetamide

*Yield based on the last step

### EXAMPLE 2

### 1-(5-chloro-1 H-benzimidazol-2-yl)-N-[3-methyl-4-(morpholin-3-on-4-yl)-phenyl]-cyclopropanecarboxamide

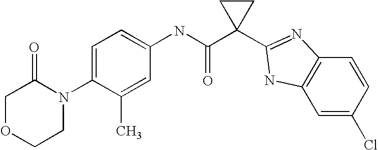 (a) monobenzyl cyclopropane-1,1-dicarboxylate

0.230 g (10 mmol) sodium are suspended in 5.00 g (46 mmol) benzylalcohol and stirred for 2 h at ambient temperature, 1 h at 60° C. and 2 h at 80° C. The cloudy solution is cooled to ambient temperature, combined with 0.851 g (5.0 mmol) 6,6-dimethyl-5,7-dioxaspiro[2,5]octan-4,8-dione and stirred for 30 min. Then it is combined with 10 ml 1N-hydrochloric acid, stirred for 30 min, made alkaline with sat. sodium bicarbonate solution, washed three times with ethyl acetate and poured onto ice/6N hydrochloric acid. The precipitated product is washed with water and dried in the spray gun over KOH.

Yield: 0.500 g (45%) R_{f} value: 0.48 (silica gel; ethyl acetate/petroleum ether=1:2) C₁₂H₁₂O₄ (220.22) Mass spectrum: (M+H)⁺=221

(b) monobenzyl N-[3-methyl-4-(morpholin-3-on-4-yl)-phenyl]-cyclopropane-1,1-dicarboxylate monoamide

A solution of 0.468 g (2.27 mmol) 3-methyl-4-(morpholin-3-on-4-yl)-aniline in 2 ml DMF is combined with 0.33 ml (3.0 mmol) N-methylmorpholine and 0.931 g (2.9 mmol) TBTU. After 5 min 0.500 g (2.27 mmol) monobenzyl cyclopropane-1,1-dicarboxylate are added and the mixture is stirred for 5.5 h at ambient temperature. Then it is combined with ice water, extracted three times with ethyl acetate and the organic phase is washed with sat. sodium bicarbonate solution, 0.5 M KHSO₄ solution, water and sat. NaCl solution and dried over MgSO₄. The crude product is further reacted directly.

Yield: 0.90 g (97%) C₂₃H₂₄N₂O₅ (408.45) Mass spectrum: (M+H)⁺=409 R_{f} value: 0.65 (silica gel; ethyl acetate/ethanol=9:1)

(c) N-[3-methyl-4-(morpholin-3-on-4-yl)-phenyl]-cyclopropane-1,1-dicarboxylic acid monoamide

0.90 g (2.20 mmol) monobenzyl N-[3-methyl-4-(morpholin-3-on-4-yl)-phenyl]-cyclopropane-1,1-dicarboxylate monoamide are hydrogenated in 10 ml of methanol and 0.10 g 10% Pd/charcoal at 5 bar hydrogen pressure for 230 min. Then the mixture is filtered and evaporated down i. vac..

Yield: 0.64 g (91%) R_{f} value: 0.25 (silica gel; ethyl acetate/ethanol 9:1+1% conc. ammonia solution) C₂₃H₂₄N₂O₅ (318.33) Mass spectrum: (M+H)⁺=319

(d) 1-(5-chloro-1H-benzimidazol-2-yl)-N-[3-methyl-4-(morpholin-3-on-4-yl)-phenyl]-cyclopropanecarboxamide

0.70 g (2.2 mmol) N-[3-methyl-4-(morpholin-3-on4-yl)-phenyl]-cyclopropane-1,1-dicarboxylic acid monoamide and 0.314 g of 4-chloro-1,2-phenylenediamine are reacted in two steps according to Example 2a/1a-ii to yield the title compound.

Yield: 0.64 g (32%) R_{f} value: 0.35 (silica gel; ethyl acetate+1% ammonia) C₂₂H₂₁ClN₄O₃ (424.88) Mass spectrum: (M−H)⁻=425/427 (chlorine isotope)

The following were prepared analogously:

| | Structural formula | | | |
|---|---|---|---|---|
| No. | Name | Yield* | Mass peak(s) | R_{f} value |
| 3 | 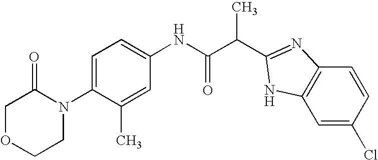 | 77% | (M + H)⁺ = 413/415 (chlorine isotope) | 0.56 (silica gel, CH₂Cl₂/C₂H₅OH 9:1) |
| | 2-(5-chloro-1H-benzimidazol-2-yl)-N-[3-methyl-4-(morpholin-3-on-4-yl)-phenyl]-propionamide | | | |
| 4 | 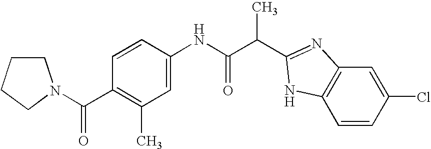 | 84% | (M + H)⁺ = 411/413 (chlorine isotope) | 0.48 (silica gel, CH₂Cl₂/C₂H₅OH 9:1) |
| | 2-(5-chloro-1H-benzimidazol-2-yl)-N-[3-methyl-4-(pyrrolidin-1-ylcarbonyl)-phenyl]-propionamide | | | |
| 6 | 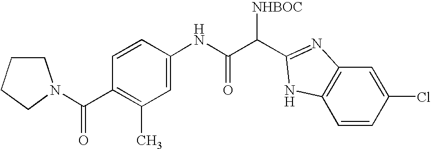 | 45% | (M + H)⁺ = 512/514 (chlorine isotope) | 0.46 (silica gel, CH₂Cl₂/C₂H₅OH 9:1) |

2-(5-chloro-1H-benzimidazol-2-yl)-N-[3-methyl-4-(pyrrolidin-1-ylcarbonyl)-phenyl]-2-tert.butoxycarbonylamino-acetamide

*Yield of the last step

### EXAMPLE 5

### 2-(5-chloro-1H-benzimidazol-2-yl)-N-[3-methyl-4-(pyrrolidin-1-ylcarbonyl)-phenyl]-2-amino-acetamide

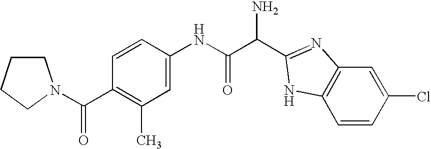

290 mg (0.566 mmol) 2-(5-chloro-1H-benzimidazol-2-yl)-N-[3-methyl-4-(pyrrolidin-1-ylcarbonyl)-phenyl]-2-tert. butoxycarbonylamino-acetamide are dissolved in 4.0 ml methylene chloride, combined with 1.0 ml TFA and stirred for 3 h at ambient temperature. After evaporation i. vac. the crude product is purified by chromatography (reversed phase silica gel RP-8; eluant: water/methanol 9:1->1:1).

Yield: 25% R_{f} value: 0.28 (RP8; methanol/5% NaCl solution=6:4) C₂₁H₂₂ClN₅O₂*2 CF₃COOH (639.931/411.89) Mass spectrum: (M+H)⁺=412/414 (chlorine isotope)

The following may also be prepared analogously to the sequences described in Examples 1, 2 and 5:
(8) 2-(5-chloro-1H-benzimidazol-2-yl)-N-[3-methyl4-(pyrrolidin-1-ylcarbonyl)-phenyl]-pent-4-enoic acid amide 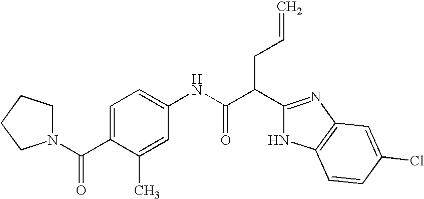
(9) 4-(5-chloro-1H-benzimidazol-2-yl)-N-[3-methyl-4-(morpholin-3-on-4-yl)-phenyl]-1-methyl-piperidine-4-carboxamide 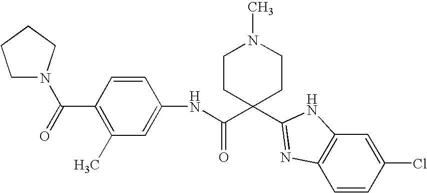
(10) 2-(5-chloro-1H-benzimidazol-2-yl)-N-[3-methyl-4-(2,5-dihydro-pyrrol-1-yl-carbonyl)-phenyl]-2-(pyrid-3-yl)-acetamide 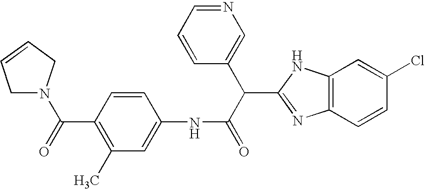
(11) 2-(5-chloro-1H-benzimidazol-2-yl)-N-[3-methyl-4-(thiazolidin-3-ylcarbonyl)-phenyl]-propionamide 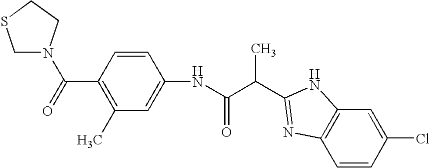
(12) 2-(5-chloro-1H-benzimidazol-2-yl)-N-[3-methyl-4-(morpholin-4-ylcarbonyl)-phenyl]-propionamide 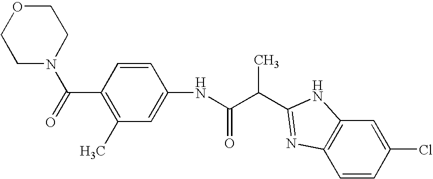
(13) 2-(5-chloro-1H-benzimidazol-2-yl)-N-[3-methyl-4-(pyrrolidin-1-ylcarbonyl)-phenyl]-4-phenyl-butanoic acid amide 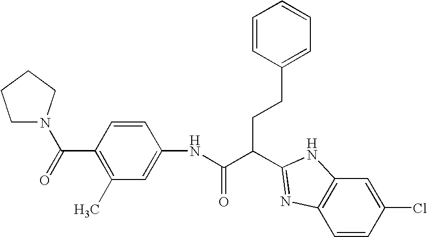
(14) 2-(5-chloro-1H-benzimidazol-2-yl)-N-[3-methyl-4-(pyrrolidin-1-ylcarbonyl)-phenyl]-5-dimethylamino-pentanoic acid amide 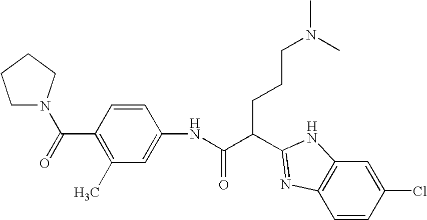
(15) 2-(5-chloro-1H-benzimidazol-2-yl)-N-[3-methyl-4-(pyrrolidin-1-ylcarbonyl)-phenyl]-4-methyl-pentanoic acid amide 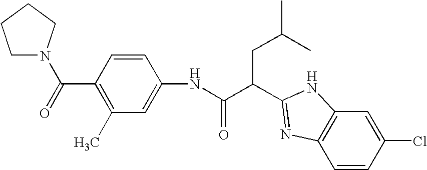
(16) 2-(5-chloro-1H-benzimidazol-2-yl)-N-[3-methyl-4-(pyrrolidin-1-ylcarbonyl)-phenyl]-2-methyl-propanoic acid amide

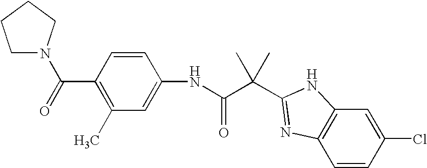

R_{f} value: 0.2 (silica gel; petroleum ether:ethyl acetate=1:9) C₂₃H₂₅ClN₄O₂ (424.92) Mass spectrum: (M+H)⁺=425/427 (chlorine isotope)
(17) 4-(5-chloro-1H-benzimidazol-2-yl)-N-[3-methyl-4-(morpholin-3-on-4-yl)-phenyl]-tetrahydropyran-4-carboxamide 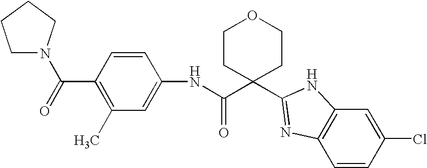
(18) 2-(5-chloro-1H-benzimidazol-2-yl)-N-[3-methyl-4-(pyrrolidin-1-ylcarbonyl)-phenyl]-4-hydroxy-butanoic acid amide 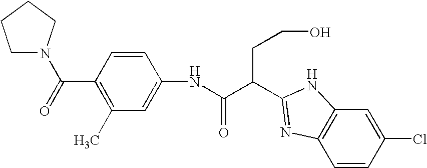
(19) 2-(5-chloro-1H-benzimidazol-2-yl)-N-[3-methoxy-4-(pyrrolidin-1-ylcarbonyl)-phenyl]-4-methoxy-butanoic acid amide 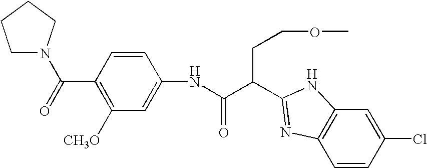
(20) 1-(5-chloro-1H-benzimidazol-2-yl)-N-[3-methyl-4-(morpholin-3-on-4-yl)-phenyl]-cyclopentanecarboxamide 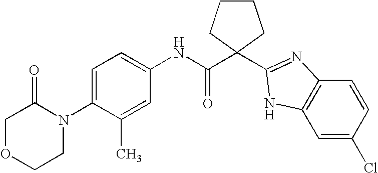
(21) 2-(5-chloro-1H-benzimidazol-2-yl)-N-[3-chloro-4-(morpholin-3-on-4-yl)-phenyl]-2-methyl-propanecarboxamide 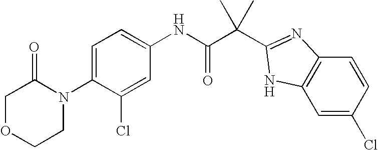
(22) 2-(5-bromo-1H-benzimidazol-2-yl)-N-[3-trifluoromethyl-4-(piperidin-2-on-1-yl)-phenyl]-propionamide 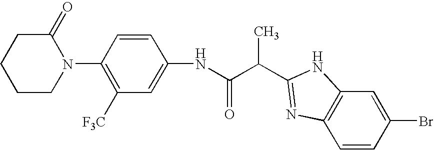
(23) 2-(5-bromo-1H-benzimidazol-2-yl)-N-[3-chloro-4-(4,4-dimethyl-oxazolidin-2-on-3-yl)-phenyl]-propionamide 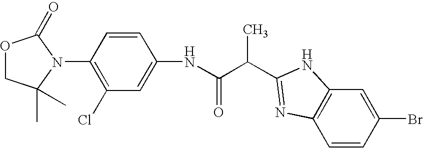
(24) 2-(5-chloro-1H-benzimidazol-2-yl)-N-[3-chloro-4([1,3]oxazepan-2-on-3-yl)-phenyl]-2-propionamide 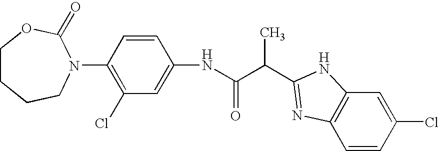
(25) 2-(5-chloro-1H-benzimidazol-2-yl)-N-[3-chloro-4([1,4]oxazepan-5-on-4-yl)-phenyl]-2-propionamide 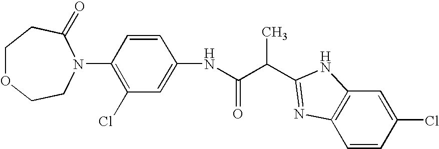
(26) 2-(5-chloro-1H-benzimidazol-2-yl)-N-[3-chloro-4-(morpholin-3-on-4-yl)-phenyl]-4-methoxy-butanecarboxamide 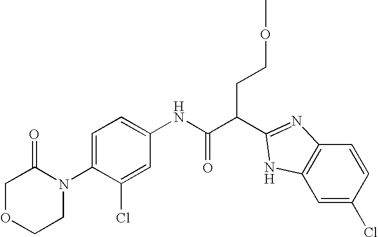
(27) 2-(5-chloro-1H-benzimidazol-2-yl)-N-[3-methyl-4-(1,1-dioxo-[1,2]thiazinan-2-yl)-phenyl]-propionamide 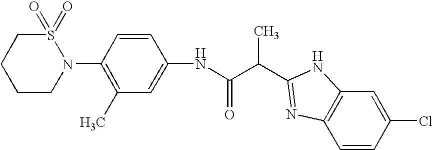
(28) 2-(5-chloro-1H-benzimidazol-2-yl)-N-[3-chloro-4-(morpholin-3-on-4-yl)-phenyl]-4-hydroxy-butanecarboxamide 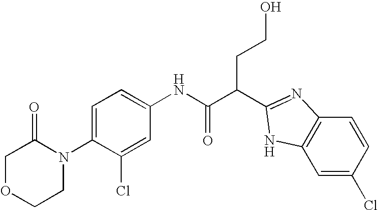
(29) 2-(5-chloro-1H-benzimidazol-2-yl)-N-[3-methyl-4-(2-methyl-pyrrolidin-1-yl-carbonyl)-phenyl]-propionamide 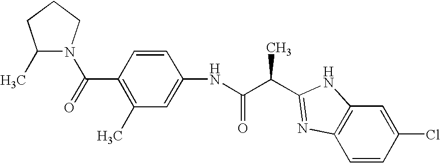
(30) 2-(5-chloro-1H-benzimidazol-2-yl)-N-[3-trifluoromethyl-4-(pyrrolidin-1-ylcarbonyl)-phenyl]-2-thiophen-3-yl-acetamide 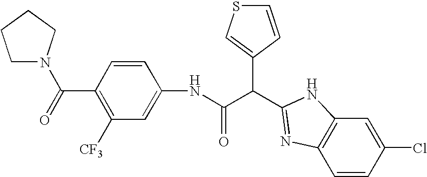
(31) 2-(5-chloro-1H-benzimidazol-2-yl)-N-[3-chloro-4-(morpholin-3-on-4-yl)-phenyl]-4-methylsulphanyl-butanecarboxamide 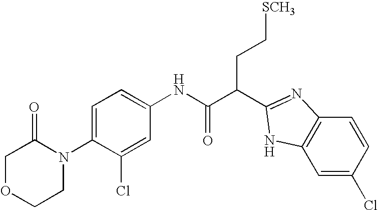
(32) 2-(5-chloro-1H-benzimidazol-2-yl)-N-[3-chloro-4-(morpholin-3-on-4-yl)-phenyl]-4-methylsulphonyl-butanecarboxamide 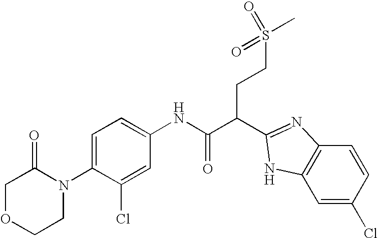
(33) 2-(5-chloro-1H-benzimidazol-2-yl)-N-[3-methyl-4-(pyrrolidin-1-ylcarbonyl)-phenyl]-4-carboxy-butanoic acid amide 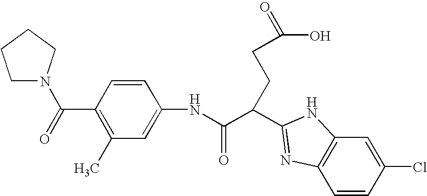
(34) 2-(5-chloro-1H-benzimidazol-2-yl)-N-[3-methyl-4-(pyrrolidin-1-ylcarbonyl)-phenyl]-4-methoxycarbonyl-butanoic acid amide 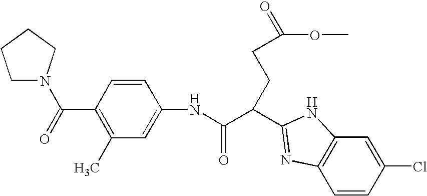
(35) 2-(5-chloro-1H-benzimidazol-2-yl)-N-[3-chloro-4-(4-methyl-[1,4]-diazepan-1-yl)-phenyl]-propanoic acid amide 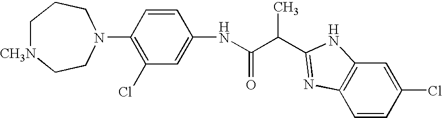
(36) 2-(5-chloro-1H-benzimidazol-2-yl)-N-[3-methyl-4-(4-methyl-[1,4]-diazepan-1-yl)-phenyl]-2-methylpropanoic acid amide 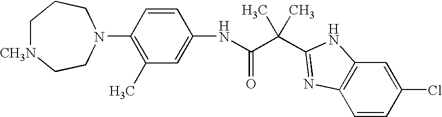
(37) 2-(5-chloro-1H-benzimidazol-2-yl)-N-[3-methyl-4-(morpholin-1-yl)-phenyl]-2-methyl-pentanoic acid amide 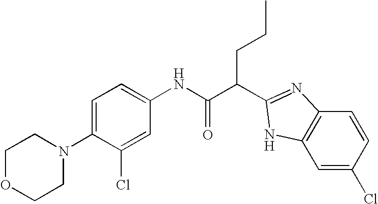
(38) 2-(5-bromo-1H-benzimidazol-2-yl)-N-[3-methyl-4-(morpholin-3-on-4-yl)-phenyl]-2-methyl-propanecarboxamide

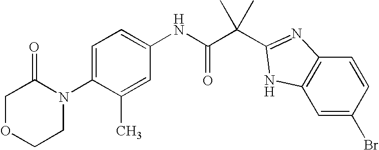

R_{f} value: 0. 15 (silica gel; petroleum ether:ethyl acetate=1:9) C₂₂H₂₃BrN₄O₃ (471.35) Mass spectrum: (M+H)⁺=471/473 (bromine isotope)
(39) 2-(5-ethynyl-1H-benzimidazol-2-yl)-N-[3-methyl-4-(morpholin-3-on-4-yl)-phenyl]-2-methyl-propanecarboxamide 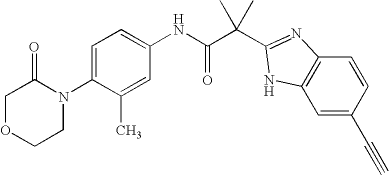
(40) 2-(5-bromo-1H-benzimidazol-2-yl)-N-[3-methyl-4-(morpholin-3-on-4-yl)-phenyl]aminocarbonyl-tetrahydrofuran

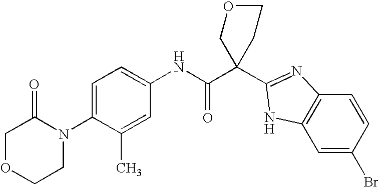

1. A compound of the formula 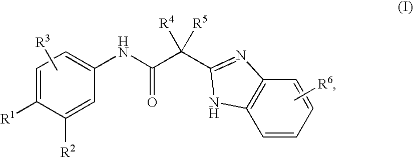
wherein
R¹ denotes a 4- to 7-membered cycloalkyleneiminocarbonyl group, wherein
the cycloalkyleneimino moiety may be substituted in the carbon skeleton by one or two fluorine atoms, one or two C₁₋₃-alkyl, C₂₋₃-alkenyl, C₂₋₃-alkynyl, hydroxy-C₁₋₃-alkyl, C₁₋₃-alkoxy-C₁₋₃-alkyl, phenyl-C₁₋₃-alkyl, heteroaryl-C₁₋₃-alkyl, amino-C₁₋₃-alkyl, C₁₋₅-alkylamino-C₁₋₃-alkyl, di-(C₁₋₅-alkyl)-amino-C₁₋₃-alkyl, a 4- to 7-membered cycloalkyleneimino-C₁₋₃-alkyl, carboxy-C₁₋₃-alkyl, C₁₋₃-alkoxycarbonyl-C₁₋₃-alkyl, aminocarbonyl-C₁₋₃-alkyl, C₁₋₃-alkylaminocarbonyl-C₁₋₃-alkyl, di-(C₁₋₃-alkyl)-aminocarbonyl-C₁₋₃-alkyl, a 4- to 7-membered cycloalkyleneiminocarbonyl-C₁₋₃-alkyl, C₁₋₅-alkoxycarbonylamino-C₁₋₃-alkyl, C₁₋₃-alkyl-carbonylamino-C₁₋₃-alkyl, C₁₋₃-alkylsulphonylamino-C₁₋₃-alkyl, carboxy, C₁₋₃-alkoxycarbonyl, aminocarbonyl, C₁₋₃-alkylaminocarbonyl, di-(C₁₋₃-alkyl)-aminocarbonyl, a 4- to 7-membered cycloalkyleneiminocarbonyl, hydroxy, C₁₋₃-alkoxy, amino, C₁₋₃-alkylamino, di-(C₁₋₃-alkyl)-amino, a 4- to 7-membered cycloalkyleneimino, a phenyl or a 5- to 6-membered heteroaryl group, with the proviso that in the substitution of a methylene group adjacent to the imino group two heteroatoms are separated from one another by at least two carbon atoms, and/or
a methylene group in the 3-position of a 5-membered cycloalkyleneimino group may be replaced by a sulphur atom, a sulphinyl or sulphonyl group, or
a methylene group in the 4-position of a 6- or 7-membered cycloalkyleneimino group may be replaced by an oxygen or sulphur atom, a carbonyl, sulphinyl or sulphonyl group or by an —NH— group optionally substituted by a C₁₋₃-alkyl, formyl or C₁₋₃-alkylcarbonyl group, while additionally a methylene group adjacent to the nitrogen atom, to which the cycloalkyleneimino group is bound, may be replaced by a carbonyl, sulphinyl or sulphonyl group, with the proviso that
in the substitution of the previously mentioned 6- to 7-membered cycloalkyleneimino groups, wherein a methylene group is replaced by an oxygen or sulphur atom, a sulphinyl or sulphonyl group, two heteroatoms are separated from one another by at least two carbon atoms,
a 5- to 7-membered cycloalkenyleneiminocarbonyl group optionally substituted by one or two C₁₋₃-alkyl, amino-C₁₋₃-alkyl, C₁₋₃-alkylamino-C₁₋₃-alkyl, di-(C₁₋₃-alkyl)-amino-C₁₋₃-alkyl, a 4- to 7-membered cyclo-alkyleneimino-C₁₋₃-alkyl, phenyl, phenyl-C₁₋₃-alkyl, heteroaryl, heteroaryl-C₁₋₃-alkyl, aminocarbonyl, C₁₋₃-alkylaminocarbonyl, di-(C₁₋₃-alkyl)-aminocarbonyl or 4- to 7-membered cycloalkyleneiminocarbonyl groups, wherein the double bond is not bound to the imino nitrogen atom,
a group of general formula 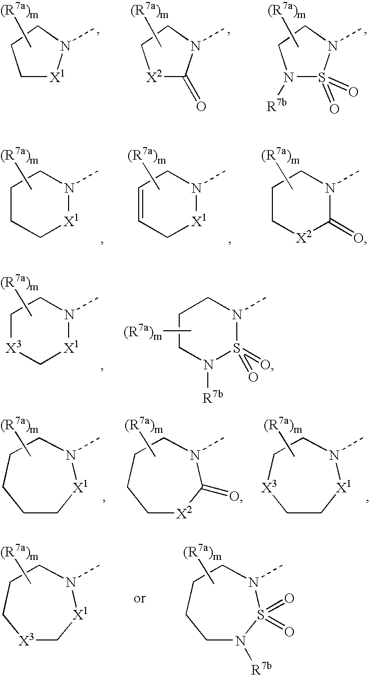
wherein
m is the number 1 or 2,
R^{7a} in each case independently of one another denotes a hydrogen or fluorine atom or a C₁₋₅-alkyl, C₁₋₅-alkoxy-C₁₋₅-alkyl, amino-C₁₋₅-alkyl, C₁₋₅-alkylamino-C₁₋₅-alkyl, di-(C₁₋₅-alkyl)-amino-C₁₋₅-alkyl, aminocarbonyl, C₁₋₅-alkylaminocarbonyl, di-(C₁₋₅-alkyl)-aminocarbonyl, hydroxy, hydroxy-C₁₋₅-alkyl, C₁₋₅-alkoxy, amino, C₁₋₅-alkylamino, di-(C₁₋₅-alkyl)-amino, or C₁₋₅-alkylcarbonylamino group while
in the above-mentioned substituted 5- to 7-membered groups R¹ the heteroatoms F, O or N optionally introduced with R^{7a} as substituents are not separated by precisely one carbon atom from a heteroatom selected from among N, O, S.
R^{7b} each independently of one another denote a hydrogen atom or a C₁₋₅-alkyl group,
R^{7c} each independently of one another denote a hydrogen atom, a C₁₋₅-alkyl, C₁₋₅-alkylcarbonyl or C₁₋₅-alkoxycarbonyl group,
X¹ denotes a carbonyl, thiocarbonyl or sulphonyl group,
X² denotes an oxygen atom or a —NR^{7b}— group,
X³ denotes an oxygen or sulphur atom or a —NR^{7c}— group,
a group of general formula 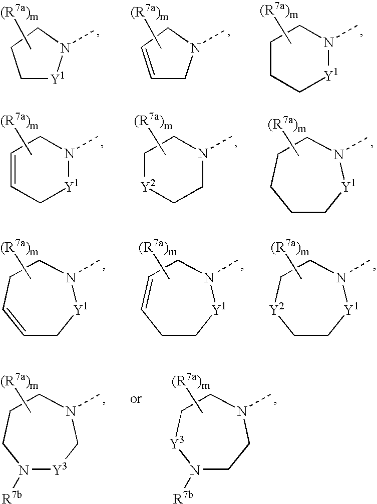
wherein
m is the number 1 or 2,
R^{7a}, R^{7b} and R^{7c} are as hereinbefore defined,
Y¹ denotes an oxygen atom or a —CH₂—, —CHR^{7b}— or
—NR^{7c}— group,
Y² denotes an oxygen or sulphur atom, an —NR^{7c}— group, and
Y³ denotes a carbonyl or sulphonyl group,
R² denotes a hydrogen, fluorine, chlorine or bromine atom, a C₁₋₃-alkyl group wherein the hydrogen atoms may be wholly or partly replaced by fluorine atoms, a C₂₋₃-alkenyl, C₂₋₃-alkynyl, C₁₋₃-alkoxy, a mono-, di- or trifluoromethoxy group,
R³ denotes a hydrogen or halogen atom or a C₁₋₃-alkyl group,
R⁴ denotes a hydrogen atom, an amino, C₁₋₅-alkylcarbonylamino, C₁₋₅-alkoxycarbonylamino, a C₂₋₃-alkenyl or C₂₋₃-alkynyl group,
a straight-chain or branched C₁₋₅-alkyl group which is optionally substituted by a fluorine atom, a mono-, di- or trifluoromethyl, a nitrile, hydroxy, a C₁₋₅-alkoxy group wherein the hydrogen atoms may be wholly or partly replaced by fluorine atoms, a mercapto, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, a 4- to 7-membered cycloalkyleneimino, C₁₋₅-alkylcarbonylamino, carboxy or C₁₋₅-alkoxycarbonyl group,
a phenyl or heteroaryl, phenyl-C₁₋₃-alkyl or heteroaryl-C₁₋₃-alkyl group which is optionally mono- or polysubstituted by fluorine, chlorine or bromine atoms, C₁₋₃-alkyl, amino, C₁₋₃-alkylamino, di-(C₁₋₃-alkyl)-amino, hydroxy, C₁₋₄-alkoxy, mono-, di- or trifluoromethoxy, carboxy or C₁₋₃-alkoxycarbonyl group,
R⁵ denotes a hydrogen atom or a C₁₋₃-alkyl group, or
R⁴ and R⁵ together with the carbon atom to which they are bound denote a C₃₋₆-cycloalkyl group, wherein
one of the methylene groups of the C₃₋₄-cycloalkyl group thus formed may be replaced by an oxygen or sulphur atom, an imino, C₁₋₃-alkylimino or acylimino group,
and
R⁶ denotes a hydrogen, fluorine, chlorine or bromine atom, a C₁₋₃-alkyl group wherein the hydrogen atoms may be wholly or partly replaced by fluorine atoms, a C₂₋₃-alkenyl or C₂₋₃-alkynyl, a hydroxy, C_{1,3}-alkoxy, trifluoromethoxy, amino, nitro or cyano group,
while, unless otherwise stated, by the term “heteroaryl group” mentioned hereinbefore in the definitions is meant a monocyclic 5- or 6-membered heteroaryl group optionally substituted in the carbon skeleton by a fluorine, chlorine, bromine or iodine atom, a C₁₋₃-alkyl, amino, C₁₋₃-alkylamino, di-(C₁₋₃-alkyl)-amino, C₁₋₃-alkoxy, carboxy, C₁₋₃-alkoxycarbonyl or C₁₋₃-alkoxycarbonylamino group, wherein
the 6-membered heteroaryl group contains one, two or three nitrogen atoms and
the 5-membered heteroaryl group contains an imino group optionally substituted by a C₁₋₃-alkyl or phenyl-C₁₋₃-alkyl group, or an oxygen or sulphur atom, or
an imino group optionally substituted by a C₁₋₃-alkyl, amino-C₂₋₃-alkyl, C₁₋₃-alkylamino-C₂₋₃-alkyl, di-(C₁₋₃-alkyl)-amino-C₂₋₃-alkyl, a 4- to 7-membered cycloalkyleneimino-C₁₋₃-alkyl or phenyl-C₁₋₃-alkyl group, or an oxygen or sulphur atom and additionally a nitrogen atom or
an imino group optionally substituted by a C₁₋₃-alkyl or phenyl-C₁₋₃-alkyl group and two or three nitrogen atoms,
and moreover a phenyl ring optionally substituted by a fluorine, chlorine or bromine atom or a C₁₋₃-alkyl, hydroxy or C₁₋₃-alkoxy group may be fused to the above-mentioned monocyclic heteroaryl groups via two adjacent carbon atoms
and the bond is effected via a nitrogen atom or a carbon atom of the heterocyclic moiety or a fused-on phenyl ring,
while the alkyl and alkoxy groups contained in the foregoing definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
or a tautomer or salt thereof.

2. A compound of the formula I according to claim 1, wherein
R¹ denotes a 4- to 7-membered cycloalkyleneiminocarbonyl group, wherein
the cycloalkyleneimino moiety may be substituted in the carbon skeleton by one or two fluorine atoms, one or two C₁₋₃-alkyl, hydroxy-C₁₋₃-alkyl, heteroaryl-C₁₋₃-alkyl, amino-C₁₋₃-alkyl, C₁₋₅-alkylamino-C₁₋₃-alkyl, di-(C₁₋₅-alkyl)-amino-C₁₋₃-alkyl, a 4- to 7-membered cycloalkyleneimino-C₁₋₃-alkyl, carboxy-C₁₋₃-alkyl, C₁₋₃-alkoxycarbonyl-C₁₋₃-alkyl, carboxy, C₁₋₃-alkoxycarbonyl, aminocarbonyl, C₁₋₃-alkylaminocarbonyl, di-(C₁₋₃-alkyl)-aminocarbonyl, a 4- to 7-membered cycloalkyleneiminocarbonyl,
hydroxy, C₁₋₃-alkoxy, amino, C₁₋₃-alkylamino, di-(C₁₋₃-alkyl)-amino, a 4- to 7-membered cycloalkyleneimino or a 5- to 6-membered heteroaryl group, with the proviso that in the substitution of a methylene group adjacent to the imino group two heteroatoms are separated from one another by at least two carbon atoms, and/or
a methylene group in the 3-position of a 5-membered cycloalkyleneimino group may be replaced by a sulphur atom or
a methylene group in the 4-position of a 6- or 7-membered cycloalkyleneimino group may be replaced by an oxygen or sulphur atom, a carbonyl, sulphinyl or sulphonyl group or by an —NH— group optionally substituted by a C₁₋₃-alkyl, formyl or C₁₋₃-alkylcarbonyl group, with the proviso that
in the substitution of the previously mentioned 6- to 7-membered cycloalkyleneimino groups, wherein a methylene group is replaced by an oxygen or sulphur atom, a sulphinyl or sulphonyl group, two heteroatoms are separated from one another by at least two carbon atoms,
a 5- to 7-membered cycloalkenyleneiminocarbonyl group optionally substituted by one or two C₁₋₃-alkyl, amino-C₁₋₃-alkyl, C₁₋₃-alkylamino-C₁₋₃-alkyl, di-(C₁₋₃-alkyl)-amino-C₁₋₃-alkyl, a 4- to 7-membered cyclo-alkyleneimino-C₁₋₃-alkyl, heteroaryl, heteroaryl-C₁₋₃-alkyl, aminocarbonyl, C₁₋₃-alkylaminocarbonyl, di-(C₁₋₃-alkyl)-aminocarbonyl or 4- to 7-membered cycloalkyleneiminocarbonyl groups, wherein the double bond is not bound to the imino nitrogen atom,
a group of general formula 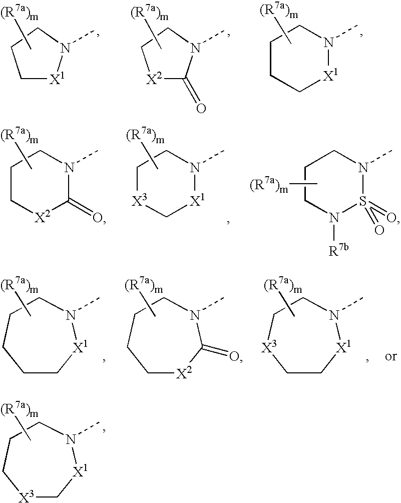
wherein
m is the number 1 or 2,
R^{7a} each independently of one another denote a hydrogen or fluorine atom or a C₁₋₅-alkyl, amino-C₁₋₅-alkyl, C₁₋₅-alkylamino-C₁₋₅-alkyl, di-(C₁₋₅-alkyl)-amino-C₁₅-alkyl, hydroxy, hydroxy-C₁₋₅-alkyl, C₁₋₅-alkoxy, amino, C₁₋₅-alkylamino or di-(C₁₅-alkyl)-amino group, while
in the above-mentioned substituted 5- to 7-membered groups R¹ the heteroatoms F, O or N optionally introduced with R^{7a} as substituents are not separated by precisely one carbon atom from a heteroatom selected from among N, O, S,
R^{7b} each independently of one another denote a hydrogen atom or a C₁₋₅-alkyl group,
R^{7c} each independently of one another denote a hydrogen atom, a C₁₋₅-alkyl or C₁₋₅-alkylcarbonyl group,
X¹ denotes a carbonyl or sulphonyl group,
X² denotes an oxygen atom or a —NR^{7b}— group,
X³ denotes an oxygen or sulphur atom or a —NR^{7c}— group,
a group of general formula 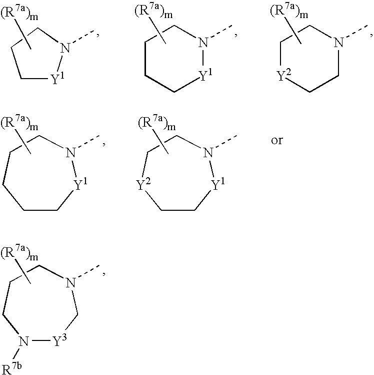
wherein
m is the number 1 or 2,
R^{7a}, R^{7b} and R^{7c} are as hereinbefore defined,
Y¹ denotes an oxygen atom or a —CH₂—, —CHR^{7b}— or —NR^{7c}— group,
Y² denotes an oxygen or sulphur atom, an —NR^{7c} group, and
Y³ denotes a carbonyl or sulphonyl group,
R² denotes a hydrogen, fluorine, chlorine or bromine atom, a C₁₋₃-alkyl group wherein the hydrogen atoms may be wholly or partly replaced by fluorine atoms,
a C₂₋₃-alkenyl, C₂₋₃-alkynyl, C₁₋₃-alkoxy, a mono-, di- or trifluoromethoxy group,
R³ denotes a hydrogen or fluorine, chlorine or bromine atom or a C₁₋₃-alkyl group,
R⁴ denotes a hydrogen atom, an amino, C₁₋₅-alkylcarbonylamino, C₁₋₅-alkoxycarbonylamino, a C₂₋₃-alkenyl or C₂₋₃-alkynyl group,
a straight-chain or branched C₁₋₅-alkyl group which is optionally substituted by a fluorine atom, a mono-, di- or trifluoromethyl, a hydroxy, a C₁₋₅-alkoxy group wherein the hydrogen atoms may be wholly or partly replaced by fluorine atoms, C₁₋₃-alkylsulphanyl, C₁₋₃-alkylsulphinyl, C₁₋₃-alkylsulphonyl, amino, C₁₋₃-alkylamino, di-(C₁₋₃-alkyl)-amino, a 4- to 7-membered cycloalkyleneimino, carboxy or C₁₋₅-alkoxycarbonyl group,
a phenyl, heteroaryl or heteroaryl-C₁₋₃-alkyl group which is optionally mono- or polysubstituted by fluorine, chlorine or bromine atoms, C₁₋₃-alkyl, amino, C₁₋₃-alkylamino, di-(C₁₋₃-alkyl)-amino, hydroxy, C₁₋₄-alkoxy, mono-, di- or trifluoromethoxy, carboxy or C₁₋₃-alkoxycarbonyl group,
R⁵ denotes a hydrogen atom or a C₁₋₃-alkyl group, or
R⁴ and R⁵ together with the carbon atom to which they are bound denote a C₃₋₆-cycloalkyl group, wherein
one of the methylene groups of a C₄₋₆-cycloalkyl group thus formed may be replaced by an oxygen or sulphur atom, an imino, C₁₋₃-alkylimino or acylimino group, and
R⁶ denotes a fluorine, chlorine or bromine atom, a methyl group wherein the hydrogen atoms may be wholly or partly replaced by fluorine atoms, an ethenyl or ethynyl, a methoxy or cyano group,
while, unless otherwise stated, by the term “heteroaryl group” mentioned hereinbefore in the definitions is meant a pyridyl, pyrazolyl, pyridazinyl, pyrimidinyl, pyrazinyl, [1,3,5]triazinyl, pyrrolyl, imidazolyl, tetrazolyl, furanyl, isoxazolyl, oxazolyl, thiophenyl, thiazolyl, isothiazolyl group optionally substituted in the carbon skeleton by a fluorine, chlorine, bromine or iodine atom, a C₁₋₃-alkyl, amino, C₁₋₃-alkylamino, di-(C₁₋₃-alkyl)-amino, C₁₋₃-alkoxy, carboxy, C₁₋₃-alkoxycarbonyl or C₁₋₃-alkoxycarbonylamino group,
while the alkyl and alkoxy groups contained in the foregoing definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
or a tautomer or salt thereof.

3. A compound of the formula I according to claim 1, wherein
R¹ denotes a 5- or 6-membered cycloalkyleneiminocarbonyl group, wherein
the cycloalkyleneimino moiety may be substituted in the carbon skeleton by one or two C₁₋₃-alkyl groups, and/or
a methylene group in the 3-position of a 5-membered cycloalkyleneimino group may be replaced by a sulphur atom, or
a methylene group in the 4-position of a 6- or 7-membered cycloalkyleneimino group may be replaced by an oxygen or
sulphur atom or by an —NH— group optionally substituted by a C₁₋₃-alkyl, formyl or C₁₋₃-alkylcarbonyl group,
a 5- or 6-membered cycloalkenyleneiminocarbonyl group optionally substituted by one or two C₁₋₃-alkyl groups, wherein the double bond is not bound to the imino nitrogen atom,
a group of general formula 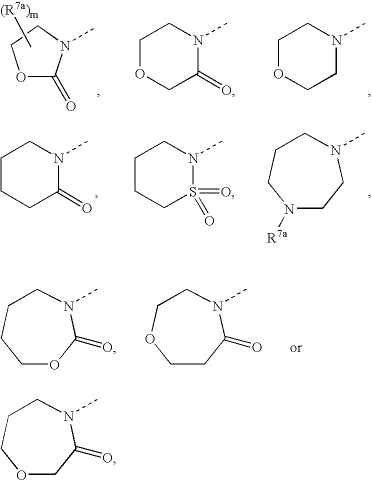
wherein
m is the number 1 or 2 and
R^{7a} each independently of one another denote a hydrogen or a C₁₋₅-alkyl group,
R² denotes a hydrogen, chlorine or bromine atom, a methyl group wherein the hydrogen atoms may be wholly or partly replaced by fluorine atoms, a methoxy, a mono-, di- or trifluoromethoxy group,
R³ denotes a hydrogen atom,
R⁴ denotes a hydrogen atom, an amino, C₁₋₅-alkylcarbonylamino, C₁₋₅-alkoxycarbonylamino, a C₂₋₃-alkenyl or C₂₋₃-alkynyl group,
a straight-chain or branched C₁₋₄-alkyl group which is optionally substituted by a mono-, di- or trifluoromethyl, a hydroxy, a methoxy group wherein the hydrogen atoms may be wholly or partly replaced by fluorine atoms, C₁₋₂-alkylsulphanyl, C₁₋₂-alkylsulphinyl, C₁₋₂-alkylsulphonyl, amino, C₁₋₂-alkylamino, di-(C₁₋₂-alkyl)-amino, a 4- to 6-membered cycloalkyleneimino, carboxy or methoxycarbonyl group,
a phenyl, pyridyl or thiophenyl group,
R⁵ denotes a hydrogen atom or a C₁₋₂-alkyl group, or
R⁴ and R⁵ together with the carbon atom to which they are bound denote a C₄₋₆-cycloalkyl group, wherein
one of the methylene groups of a C₄₋₆-cycloalkyl group thus formed may be replaced by an oxygen or sulphur atom, an imino, C₁₋₃-alkylimino or acylimino group, and
R⁶ denotes a chlorine or bromine atom or an ethynyl group,
while the alkyl and alkoxy groups contained in the foregoing definitions which have more than two carbon atoms may, unless otherwise stated, be straight-chain or branched and the alkyl groups in the previously mentioned dialkylated groups, for example the dialkylamino groups, may be identical or different,
and the hydrogen atoms of the methyl or ethyl groups contained in the foregoing definitions may be wholly or partly replaced by fluorine atoms,
or a tautomer or salt thereof.

4. A compound selected from the group consisting of:
(1) 2-(5-chloro-1H-benzimidazol-2-yl)-N-[3-methyl-4-(morpholin-3-on-4-yl)-phenyl]-2-phenyl-acetamide,
(2) 1-(5-chloro-1H-benzimidazol-2-yl)-N-[3-methyl-4-(morpholin-3-on4-yl)-phenyl]-cyclopropanecarboxamide,
(3) 2-(5-chloro-1H-benzimidazol-2-yl)-N-[3-methyl-4-(morpholin-3-on-4-yl)-phenyl]-propionamide,
(4) 2-(5-chloro-1H-benzimidazol-2-yl)-N-[3-methyl-4-(pyrrolidin-1-ylcarbonyl)-phenyl]-propionamide,
(5) 2-(5-chloro-1H-benzimidazol-2-yl)-N-[3-methyl-4-(pyrrolidin-1-ylcarbonyl)-phenyl]-2-amino-acetamide,
(6) 2-(5-chloro-1H-benzimidazol-2-yl)-N-[3-methyl-4-(pyrrolidin-1-ylcarbonyl)-phenyl]-2-tert.butoxycarbonylamino-acetamide,
(7) 2-(5-chloro-1H-benzimidazol-2-yl)-N-[3-methyl-4-(pyrrolidin-1-ylcarbonyl)-phenyl]-2-phenyl-acetamide,
(8) 2-(5-chloro-1H-benzimidazol-2-yl)-N-[3-methyl-4-(pyrrolidin-1-ylcarbonyl)-phenyl]-pent-4-enoic acid amide,
(9) 4-(5-chloro-1H-benzimidazol-2-yl)-N-[3-methyl-4-(morpholin-3-on-4-yl)-phenyl]-1-methyl-piperidine-4-carboxamide,
(10) 2-(5-chloro-1H-benzimidazol-2-yl)-N-[3-methyl-4-(2,5-dihydro-pyrrol-1-yl-carbonyl)-phenyl]-2-(pyrid-3-yl)-acetamide,
(11) 2-(5-chloro-1H-benzimidazol-2-yl)-N-[3-methyl-4-(thiazolidin-3-ylcarbonyl)-phenyl]-propionamide,
(12) 2-(5-chloro-1H-benzimidazol-2-yl)-N-[3-methyl-4-(morpholin-4-ylcarbonyl)-phenyl]-propionamide,
(13) 2-(5-chloro-1H-benzimidazol-2-yl)-N-[3-methyl-4-(pyrrolidin-1-ylcarbonyl)-phenyl]4-phenyl-butanoic acid amide,
(14) 2-(5-chloro-1H-benzimidazol-2-yl)-N-[3-methyl-4-(pyrrolidin-1-ylcarbonyl)-phenyl]-5-dimethylamino-pentanoic acid amide,
(15) 2-(5-chloro-1H-benzimidazol-2-yl)-N-[3-methyl-4-(pyrrolidin-1-ylcarbonyl)-phenyl]-4-methyl-pentanoic acid amide,
(16) 2-(5-chloro-1H-benzimidazol-2-yl)-N-[3-methyl-4-(pyrrolidin-1-ylcarbonyl)-phenyl]-2-methyl-propanoic acid amide,
(17) 4-(5-chloro-1H-benzimidazol-2-yl)-N-[3-methyl-4-(morpholin-3-on-4-yl)-phenyl]-tetrahydropyran-4-carboxamide,
(18) 2-(5-chloro-1H-benzimidazol-2-yl)-N-[3-methyl-4-(pyrrolidin-1-ylcarbonyl)-phenyl]-4-hydroxy-butanoic acid amide,
(19) 2-(5-chloro-1H-benzimidazol-2-yl)-N-[3-methoxy-4-(pyrrolidin-1-ylcarbonyl)-phenyl]-4-methoxy-butanoic acid amide,
(20) 1-(5-chloro-1H-benzimidazol-2-yl)-N-[3-methyl-4-(morpholin-3-on-4-yl)-phenyl]-cyclopentanecarboxamide,
(21) 2-(5-chloro-1H-benzimidazol-2-yl)-N-[3-chloro-4-(morpholin-3-on-4-yl)-phenyl]-2-methyl-propanecarboxamide,
(22) 2-(5-bromo-1H-benzimidazol-2-yl)-N-[3-trifluoromethyl-4-(piperidin-2-on-1-yl)-phenyl]-propionamide,
(23) 2-(5-bromo-1H-benzimidazol-2-yl)-N-[3-chloro-4-(4,4-dimethyl-oxazolidin-2-on-3-yl)-phenyl]-propionamide,
(24) 2-(5-chloro-1H-benzimidazol-2-yl)-N-[3-chloro-4([1,3]oxazepan-2-on-3-yl)-phenyl]-2-propionamide,
(25) 2-(5-chloro-1H-benzimidazol-2-yl)-N-[3-chloro-4([1,4]oxazepan-5-on-4-yl)-phenyl]-2-propionamide,
(26) 2-(5-chloro-1H-benzimidazol-2-yl)-N-[3-chloro-4-(morpholin-3-on-4-yl)-phenyl]4-methoxy-butanecarboxamide,
(27) 2-(5-chloro-1H-benzimidazol-2-yl)-N-[3-methyl-4-(1,1-dioxo-[1,2]thiazinan-2-yl)-phenyl]-propionamide,
(28) 2-(5-chloro-1H-benzimidazol-2-yl)-N-[3-chloro-4-(morpholin-3-on-4-yl)-phenyl]-4-hydroxy-butanecarboxamide,
(29) 2-(5-chloro-1H-benzimidazol-2-yl)-N-[3-methyl-4-(2-methyl-pyrrolidin-1-yl-carbonyl)-phenyl]-propionamide,
(30) 2-(5-chloro-1H-benzimidazol-2-yl)-N-[3-trifluoromethyl-4-(pyrrolidin-1-ylcarbonyl)-phenyl]-2-thiophen-3-yl-acetamide,
(31) 2-(5-chloro-1H-benzimidazol-2-yl)-N-[3-chloro-4-(morpholin-3-on-4-yl)-phenyl]-4-methylsulphanyl-butanecarboxamide,
(32) 2-(5-chloro-1H-benzimidazol-2-yl)-N-[3-chloro-4-(morpholin-3-on-4-yl)-phenyl]-4-methylsulphonyl-butanecarboxamide,
(33) 2-(5-chloro-1H-benzimidazol-2-yl)-N-[3-methyl-4-(pyrrolidin-1-ylcarbonyl)-phenyl]-4-carboxy-butanoic acid amide,
(34) 2-(5-chloro-1H-benzimidazol-2-yl)-N-[3-methyl-4-(pyrrolidin-1-ylcarbonyl)-phenyl]-4-methoxycarbonyl-butanoic acid amide,
(35) 2-(5-chloro-1H-benzimidazol-2-yl)-N-[3-chloro-4-(4-methyl-[1,4]-diazepan-1-yl)-phenyl]-propanoic acid amide,
(36) 2-(5-chloro-1H-benzimidazol-2-yl)-N-[3-methyl-4-(4-methyl-[1,4]-diazepan-1-yl)-phenyl]-2-methylpropanoic acid amide,
(37) 2-(5-chloro-1H-benzimidazol-2-yl)-N-[3-methyl-4-(morpholin-1-yl)-phenyl]-2-methyl-pentanoic acid amide,
(38) 2-(5-bromo-1H-benzimidazol-2-yl)-N-[3-methyl-4-(morpholin-3-on-4-yl)-phenyl]-2-methyl-propanecarboxamide,
(39) 2-(5-ethynyl-1H-benzimidazol-2-yl)-N-[3-methyl-4-(morpholin-3-on-4-yl)-phenyl]-2-methyl-propanecarboxamide, and
(40) 2-(5-bromo-1H-benzimidazol-2-yl)-N-[3-methyl-4-(morpholin-3-on-4-yl)-phenyl]aminocarbonyl-tetrahydrofuran,
or a tautomer or salt thereof.

5. A physiologically acceptable salt of a compound according to claim 1, 2, 3 or 4.

6. A pharmaceutical composition comprising a compound according to claim 1, 2, 3 or 4 or a physiologically acceptable salt thereof, together with one or more inert carriers and/or diluents.

7. A method for treating thrombotic disease which comprises administering to a host suffering from a thrombus or prone to thrombus formation an antithrombotic amound of a a compound according to claim 1, 2, 3 or 4 or a physiologically acceptable salt thereof.
